(19) United States
(12) Patent Application Publication
(10) Pub. No.: US 2015/0057443 A1
(43) Pub. Date: Feb. 26, 2015

(54) Cycloalkyl Amine Compounds

(71) Applicant: Prexa Pharmaceuticals, Inc., Boston, MA (US)

(72) Inventors: Philip Huxley, Oxon (GB); Jonathan R. Heal, Cambridge (GB); Richard S. Todd, Slough (GB)

(21) Appl. No.: 14/465,068

(22) Filed: Aug. 21, 2014

Related U.S. Application Data
(60) Provisional application No. 61/868,491, filed on Aug. 21, 2013.

(51) Int. Cl. C07D 295/037; C07D 211/38; C07D 491/08; C07C 215/40; C07D 211/60; C07D 211/42; C07D 295/073; C07D 295/084; C07D 221/22; C07D 265/30

(52) U.S. Cl. CPC C07D 295/037; C07D 221/22; C07D 211/38; C07D 491/08; C07D 265/30; C07D 211/60; C07D 211/42; C07D 295/073; C07D 295/084; C07C 215/40; C07C 2101/04; C07C 2101/02
USPC 540/609; 546/192; 546/112; 548/452; 546/240; 546/205; 548/574; 564/442; 548/453; 544/170; 546/215; 546/216; 546/237

(57) ABSTRACT

Cycloalkyl amine compounds of Formula (I),

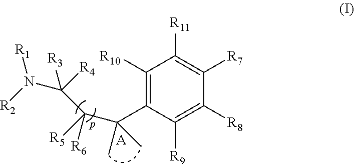

wherein ring A is C₃-C₆ cycloalkyl, optionally substituted with one or more C₁-C₃ alkyl, and R₅ is OR_{S2}, in which R_{S2} is H or C₁-C₆ alkyl, or R₅ and R₆, together with the carbon atom to which they are attached, form C═O, for use in treating CNS disorders, including movement disorders, depressive disorders, sleep disorders, cognitive dysfunctions, obesity, sexual dysfunction and substance abuse.

### RELATED APPLICATION

This application claims priority to U.S. Provisional Application Ser. No. 61/868,491, filed Aug. 21, 2013, the disclosure of which is hereby incorporated by reference in its entirety herein.

### BACKGROUND OF THE DISCLOSURE

Neuronal signals are transmitted between cells at specialized sites of contact known as synapses. The signals are generally transmitted across synapses by diffusion of soluble neurotransmitter molecules from a presynaptic cell to a postsynaptic cell. Release of neurotransmitters is triggered by a change of electrical potential in the presynaptic cell. The neurotransmitters rapidly diffuse across the synaptic cleft and provoke an electrical change in the postsynaptic cell by binding to neurotransmitter-gated ion channels. Excess neurotransmitters are rapidly removed from the synaptic cleft, either by specific enzymes or by reuptake into the presynaptic cell or surrounding glial cells. Reuptake is mediated by a variety of neurotransmitter transporters. Rapid removal ensures both spatial and temporal precision of signaling at a synapse. For example, rapid reuptake can prevent excess neurotransmitters from influencing neighboring cells and can clear the synaptic cleft before the next pulse of neurotransmitter release so that the timing of repeated, rapid signaling events is accurately communicated to the postsynaptic cell.

An imbalance of neurotransmitters in the brain can occur when not enough neurotransmitter is made and released from presynaptic cells or when the reuptake of neurotransmitters by presynaptic cells is too rapid. If neurotransmitters such as serotonin, norepinephrine, or dopamine are not made and released in effective amounts or are cleared from the synaptic cleft too quickly, then cell-to-cell communication can be affected. Clinical manifestations of such imbalances include cognitive disorders (for example, ADHD), sleep disorders, substance abuse, depression and related anxiety disorders, cognitive and movement disorders.

### SUMMARY OF THE DISCLOSURE

The present disclosure provides novel reuptake inhibitors which preferentially block the reuptake of dopamine and norepinephrine into presynaptic cells. This inhibition of neurotransmitter reuptake can increase the amount of neurotransmitter present in the synapse, thus helping to normalize the transmission of neuronal signals. Such normalization of neurotransmitter levels, particularly within the prefrontal cortex, may be useful in the treatment of central nervous system (“CNS”) disorders, such as ADHD, depression and other disturbances of affect, disturbances in appetite regulation and obesity, excessive daytime sleepiness, substance use disorders, and neurocognitive dysfunction resulting from neurodegeneration, trauma, or psychiatric conditions.

In one aspect, the present disclosure features a cycloalkyl amine compound of Formula (I) or a pharmaceutically acceptable salt or ester thereof:

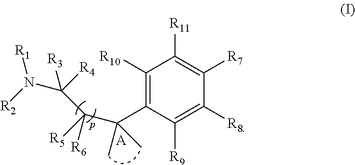

In this formula,

ring A is C₃-C₆ cycloalkyl optionally substituted with one or more C₁-C₃ alkyl;

each of R₁ and R₂ independently is H or R_{S1}, in which R_{S1} is C₁-C₆ alkyl, C₂-C₆ alkenyl, C₂-C₆ alkynyl, or C₃-C₈ cycloalkyl, and R_{S1} is optionally substituted with one or more substituents selected from the group consisting of halo, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl; and at least one of R₁ and R₂ is not H; or

R₁ and R₂, together with the nitrogen atom to which they are attached, form a 4 to 12-membered saturated heterocycloalkyl ring having 0 to 2 additional heteroatoms, and the 4 to 12-membered saturated heterocycloalkyl ring is optionally substituted with one or more substituents selected from the group consisting of halo, hydroxyl, cyano, C₁-C₆ alkyl, C₁-C₆ haloalkyl, C₂-C₆ alkenyl, C₂-C₆ alkynyl, C₁-C₆ alkoxyl, C₁-C₆ haloalkoxyl, amino, mono-C₁-C₆ alkylamino, di-C₁-C₆ alkylamino, C₃-C₅ cycloalkyl, or 4 to 12-membered heterocycloalkyl;

each of R₃ and R₄ independently, is H, C₁-C₆ alkyl, C₂-C₆ alkenyl, or C₂-C₆ alkynyl; or

R₃ and R₄, together with the carbon atom to which they are attached, form C₃-C₆ cycloalkyl;

R₅ is OR_{S2}, in which R_{S2} is H or C₁-C₆ alkyl;

R₆ is H or C₁-C₆ alkyl; or

R₅ and R₆, together with the carbon atom to which they are attached, form C═O;

each of R₇, R₈, R₉, R₁₀, and R₁₁, independently, is -Q-T, in which Q is a bond or C₁-C₃ alkyl linker optionally substituted with halo, cyano, hydroxyl or C₁-C₆ alkoxy, and T is H, halo, hydroxyl, C(O)OH, cyano, azido, or R_{S3}, in which R_{S3} is C₁-C₃ alkyl, C₂-C₆ alkenyl, C₂-C₆ alkynyl, C₁-C₆ alkoxyl, C₁-C₆ thioalkyl, C(O)OC₁-C₆ alkyl, C(O)NH₂, SO₂C₁-C₆ alkyl, SO₂C₆-C₁₀ aryl, SO₂NH₂, C(O)NH(C₁-C₆ alkyl), C(O)N(C₁-C₆ alkyl)₂, SO₂NH(C₁-C₆ alkyl), SO₂N(C₁-C₆ alkyl)₂, C₃-C₅ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl; or

R₇ and R₈, together with the carbon atoms to which they are attached, form phenyl or a 5- or 6-membered heteroaryl having 1 to 3 heteroatoms; or R₇ and R₁₁, together with the carbon atoms to which they are attached, form phenyl or a 5- or 6-membered heteroaryl having 1 to 3 heteroatoms; or R₈ and R₉, together with the carbon atoms to which they are attached, form phenyl or a 5- or 6-membered heteroaryl having 1 to 3 heteroatoms; or R₁₀ and R₁₁, together with the carbon atoms to which they are attached, form phenyl or a 5- or 6-membered heteroaryl having 1 to 3 heteroatoms; and

p is 0 or 1, provided that

(i) when p is 0, then ring A is optionally substituted C₃-C₅ cycloalkyl or substituted cyclohexyl, wherein when ring A is unsubstituted cyclobutyl, then both R₃ and R₄ are H;

(ii) when p is 0 and NR₁R₂ is unsubstituted piperidin-1-yl, then ring A is unsubstituted cyclobutyl or substituted C₃-C₆ cycloalkyl, or at least one of R₇, R₈, R₉, R₁₀, and R₁₁ is not H; and

(iii) when p is 1 and ring A is unsubstituted cyclobutyl, then R₅ is OR_{S2}, or R₅ and R₆, together with the carbon atom to which they are attached, form C═O.

One subset of the compounds of Formula (I) includes those of Formula (Ia):

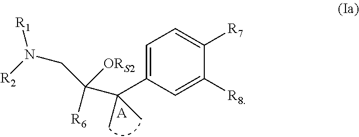

Another subset of the compounds of Formula (I) includes those of Formula (Ib) in which p is 0.

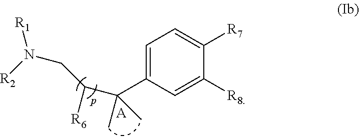

In the compounds of Formula (Ib), A may suitably be substituted or unsubstituted cyclobutyl.

Another subset of the compounds of Formula (I) includes those of Formula (Ic).

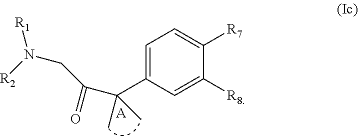

The variables in any of Formulae (Ia), (Ib), and (Ic), such as ring A, R_{S2}, R₁, R₂, R₆R₇, R₈, and p are generally as defined herein for Formula (I).

The present disclosure also provides pharmaceutical compositions comprising one or more pharmaceutically acceptable carriers and one or more compounds selected from those of any Formula disclosed herein.

The present disclosure also provides a kit comprising one or more compounds selected from those of any Formula disclosed herein or a pharmaceutically acceptable salt thereof, a container, and instructions for use.

Another aspect of this disclosure is a method of treating or preventing a CNS disorder. The method includes administering to a subject in need thereof a therapeutically effective amount of one or more compounds selected from those of any Formula disclosed herein.

Unless otherwise stated, any description of a method of treatment includes uses of the compounds to provide such treatment or prophylaxis as is described in the specification, as well as uses of the compounds to prepare a medicament to treat or prevent such condition. The treatment includes treatment of human or non-human animals including rodents and other disease models.

Further, the compounds or methods described herein can be used for research and other non-therapeutic purposes.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed disclosure. In the case of conflict, the present specification, including definitions, will control. In addition, the materials, methods and examples are illustrative only and are not intended to be limiting.

Other features and advantages of the disclosure will be apparent from the following detailed description and claims.

### DETAILED DESCRIPTION OF THE DISCLOSURE

The present disclosure provides novel cycloalkyl amine compounds, synthetic methods for making the compounds, pharmaceutical compositions containing them and various uses of the compounds.

### 1. CYCLOALKYL AMINE COMPOUNDS

The present disclosure provides the compounds of Formula (I):

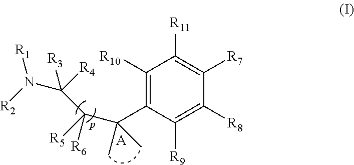

In this formula,

ring A is C₃-C₆ cycloalkyl optionally substituted with one or more C₁-C₃ alkyl;

each of R₁ and R₂ independently, is H or R_{S1}, in which R_{S1} is C₁-C₆ alkyl, C₂-C₆ alkenyl, C₂-C₆ alkynyl, or C₃-C₈ cycloalkyl, and R_{S1} is optionally substituted with one or more substituents selected from the group consisting of halo, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl; and at least one of R₁ and R₂ is not H; or

R₁ and R₂, together with the nitrogen atom to which they are attached, form a 4 to 12-membered saturated heterocycloalkyl ring having 0 to 2 additional heteroatoms, and the 4 to 12-membered saturated heterocycloalkyl ring is optionally substituted with one or more substituents selected from the group consisting of halo, hydroxyl, cyano, C₁-C₆ alkyl, C₁-C₆ haloalkyl, C₂-C₆ alkenyl, C₂-C₆ alkynyl, C₁-C₆ alkoxyl, C₁-C₆ haloalkoxyl, amino, mono-C₁-C₆ alkylamino, di-C₁-C₆ alkylamino, C₃-C₈ cycloalkyl, or 4 to 12-membered heterocycloalkyl;

each of R₃ and R₄ independently, is H, C₁-C₆ alkyl, C₂-C₆ alkenyl, or C₂-C₆ alkynyl; or

R₃ and R₄, together with the carbon atom to which they are attached, form C₃-C₆ cycloalkyl;

R₅ is OR_{S2}, in which R_{S2} is H or C₁-C₆ alkyl;

R₆ is H or C₁-C₆ alkyl; or

R₅ and R₆, together with the carbon atom to which they are attached, form C═O;

each of R₇, R₈, R₉, R₁₀, and R₁₁, independently, is -Q-T, in which Q is a bond or C₁-C₃ alkyl linker optionally substituted with halo, cyano, hydroxyl or C₁-C₆ alkoxy, and T is H, halo, hydroxyl, C(O)OH, cyano, azido, or R_{S3}, in which R_{S3} is C₁-C₃ alkyl, C₂-C₆ alkenyl, C₂-C₆ alkynyl, C₁-C₆ alkoxyl, C₁-C₆ thioalkyl, C(O)OC₁-C₆ alkyl, C(O)NH₂, SO₂C₁-C₆ alkyl, SO₂C₆-C₁₀ aryl, SO₂NH₂, C(O)NH(C₁-C₆ alkyl), C(O)N(C₁-C₆ alkyl)₂, SO₂NH(C₁-C₆ alkyl), SO₂N(C₁-C₆ alkyl)₂, C₃-C₈ cycloalkyl, C₆-C₁₀ aryl, C₆-C₁₀ aryloxy, amino, mono-C₁-C₆ alkylamino, C₆ alkylamino, 4 to 12-membered heterocycloalkyl, or 5- or 6-membered heteroaryl; or

R₇ and R₈, together with the carbon atoms to which they are attached, form phenyl or a 5- or 6-membered heteroaryl having 1 to 3 heteroatoms; or R₇ and R₁₁, together with the carbon atoms to which they are attached, form phenyl or a 5- or 6-membered heteroaryl having 1 to 3 heteroatoms; or R₈ and R₉, together with the carbon atoms to which they are attached, form phenyl or a 5- or 6-membered heteroaryl having 1 to 3 heteroatoms; or R₁₀ and R₁₁, together with the carbon atoms to which they are attached, form phenyl or a 5- or 6-membered heteroaryl having 1 to 3 heteroatoms; and

p is 0 or 1, provided that

(i) when p is 0, then ring A is optionally substituted C₃-C₅ cycloalkyl or substituted cyclohexyl, wherein when ring A is unsubstituted cyclobutyl, then both R₃ and R₄ are H;

(ii) when p is 0 and NR₁R₂ is unsubstituted piperidin-1-yl, then ring A is unsubstituted cyclobutyl or substituted C₃-C₆ cycloalkyl, or at least one of R₇, R₈, R₉, R₁₀, and R₁₁ is not H; and

(iii) when p is 1 and ring A is unsubstituted cyclobutyl, then R₅ is OR_{S2}, or R₅ and R₆, together with the carbon atom to which they are attached, form C═O.

Suitably in some embodiments R₃ and R₄ are both H.

Similarly, R₉, R₁₀ and R₁₁ may all be H.

In some subsets of the compounds of the disclosure, p is preferably 1.

Thus, the present disclosure provides the compounds of Formula (Ia)

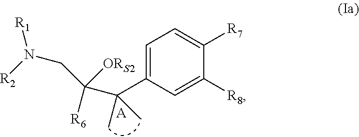

or a pharmaceutically acceptable salt or ester thereof, wherein

ring A is C₃-C₆ cycloalkyl optionally substituted with one or more C₁-C₃ alkyl;

each of R₁ and R₂ independently, is H or R_{S1}, in which R_{S1} is C₁-C₆ alkyl, C₂-C₆ alkenyl, C₂-C₆ alkynyl, or C₃-C₅ cycloalkyl, and R_{S1} is optionally substituted with one or more substituents selected from the group consisting of halo, hydroxyl, cyano, C₁-C₆ alkoxyl, amino, mono-C₁-C₆ alkylamino, di-C₁-C₆ alkylamino, C₃-C₅ cycloalkyl, C₆-C₁₀ aryl, 4 to 12-membered heterocycloalkyl, and 5- or 6-membered heteroaryl; and at least one of R₁ and R₂ is not H; or

R₁ and R₂, together with the nitrogen atom to which they are attached, form a 4 to 12-membered saturated heterocycloalkyl ring having 0 to 2 additional heteroatoms, and the 4 to 12-membered saturated heterocycloalkyl ring is optionally substituted with one or more substituents selected from the group consisting of halo, hydroxyl, cyano, C₁-C₆ alkyl, C₁-C₆ haloalkyl, C₂-C₆ alkenyl, C₂-C₆ alkynyl, C₁-C₆ alkoxyl, C₁-C₆ haloalkoxyl, amino, mono-C₁-C₆ alkylamino, di-C₁-C₆ alkylamino, C₃-C₈ cycloalkyl, or 4 to 12-membered heterocycloalkyl;

R_{S2} is H or C₁-C₆ alkyl;

R₆ is H or C₁-C₆ alkyl; and

each of R₇ and R₈, independently, is -Q-T, in which Q is a bond or C₁-C₃ alkyl linker optionally substituted with halo, cyano, hydroxyl or C₁-C₆ alkoxy, and T is H, halo, hydroxyl, C(O)OH, cyano, azido, or R_{S3}, in which R_{S3} is C₁-C₃ alkyl, C₂-C₆ alkenyl, C₂-C₆ alkynyl, C₁-C₆ alkoxyl, C₁-C₆ thioalkyl, C(O)OC₁-C₆ alkyl, C(O)NH₂, SO₂C₁-C₆ alkyl, SO₂C₆-C₁₀ aryl, SO₂NH₂, C(O)NH(C₁-C₆ alkyl), C(O)N(C₁-C₆ alkyl)₂, SO₂NH(C₁-C₆ alkyl), SO₂N(C₁-C₆ alkyl)₂, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl; or

R₇ and R₈, together with the carbon atoms to which they are attached, form phenyl or a 5- or 6-membered heteroaryl having 1 to 3 heteroatoms.

In Formula (Ia), R_{S2} is preferably H.

The present disclosure also provides the compounds of Formula (Ib):

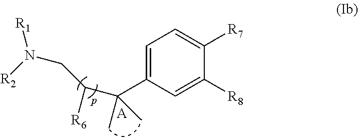

or pharmaceutically acceptable salts or esters thereof, wherein

ring A is C₃-C₅ cycloalkyl optionally substituted with one or more C₁-C₃ alkyl or cyclohexyl substituted with one or more C₁-C₃ alkyl;

each of R₁ and R₂ independently, is H or R_{S1}, in which R_{S1} is C₁-C₆ alkyl, C₂-C₆ alkenyl, C₂-C₆ alkynyl, or C₃-C₈ cycloalkyl, and R_{S1} is optionally substituted with one or more substituents selected from the group consisting of halo, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl; and at least one of R₁ and R₂ is not H; or

R₁ and R₂, together with the nitrogen atom to which they are attached, form a 4 to 12-membered saturated heterocycloalkyl ring having 0 to 2 additional heteroatoms, and the 4 to 12-membered saturated heterocycloalkyl ring is optionally substituted with one or more substituents selected from the group consisting of halo, hydroxyl, cyano, C₁-C₆ alkyl, C₁-C₆ haloalkyl, C₂-C₆ alkenyl, C₂-C₆ alkynyl, C₁-C₆ alkoxyl, C₁-C₆ haloalkoxyl, amino, mono-C₁-C₆ alkylamino, di-C₁-C₆ alkylamino, C₃-C₈ cycloalkyl, or 4 to 12-membered heterocycloalkyl;

R₆ is H or C₁-C₆ alkyl;

each of R₇ and R₈, independently, is -Q-T, in which Q is a bond or C₁-C₃ alkyl linker optionally substituted with halo, cyano, hydroxyl or C₁-C₆ alkoxy, and T is H, halo, hydroxyl, C(O)OH, cyano, azido, or R_{S3}, in which R_{S3} is C₁-C₃ alkyl, C₂-C₆ alkenyl, C₂-C₆ alkynyl, C₁-C₆ alkoxyl, C₁-C₆ thioalkyl, C(O)OC₁-C₆ alkyl, C(O)NH₂, SO₂C₁-C₆ alkyl, SO₂C₆-C₁₀ aryl, SO₂NH₂, C(O)NH(C₁-C₆ alkyl), C(O)N(C₁-C₆ alkyl)₂, SO₂NH(C₁-C₆ alkyl), SO₂N(C₁-C₆ alkyl)₂, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl; or

R₇ and R₈, together with the carbon atoms to which they are attached, form phenyl or a 5- or 6-membered heteroaryl having 1 to 3 heteroatoms; and

p is 0, provided that

when NR₁R₂ is unsubstituted piperidin-1-yl, then ring A is unsubstituted cyclobutyl or substituted C₃-C₆ cycloalkyl, or at least one of R₇ and R₈ is not H.

In some embodiments, A may be substituted or unsubstituted cyclobutyl in compounds of Formula (Ib).

The present disclosure also provides the compounds of Formula (Ic) in which p is 1:

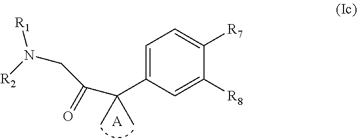

or pharmaceutically acceptable salts or esters thereof, wherein

ring A is C₃-C₆ cycloalkyl optionally substituted with one or more C₁-C₃ alkyl;

each of R₁ and R₂ independently, is H or R_{S1}, in which R_{S1} is C₁-C₆ alkyl, C₂-C₆ alkenyl, C₂-C₆ alkynyl, or C₃-C₈ cycloalkyl, and R_{S1} is optionally substituted with one or more substituents selected from the group consisting of halo, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl; and at least one of R₁ and R₂ is not H; or

R₁ and R₂, together with the nitrogen atom to which they are attached, form a 4 to 12-membered saturated heterocycloalkyl ring having 0 to 2 additional heteroatoms, and the 4 to 12-membered saturated heterocycloalkyl ring is optionally substituted with one or more substituents selected from the group consisting of halo, hydroxyl, cyano, C₁-C₆ alkyl, C₁-C₆ haloalkyl, C₂-C₆ alkenyl, C₂-C₆ alkynyl, C₁-C₆ alkoxyl, C₁-C₆ haloalkoxyl, amino, mono-C₁-C₆ alkylamino, di-C₁-C₆ alkylamino, C₃-C₈ cycloalkyl, or 4 to 12-membered heterocycloalkyl; and

each of R₇ and R₈, independently, is -Q-T, in which Q is a bond or C₁-C₃ alkyl linker optionally substituted with halo, cyano, hydroxyl or C₁-C₆ alkoxy, and T is H, halo, hydroxyl, C(O)OH, cyano, azido, or R_{S3}, in which R_{S3} is C₁-C₃ alkyl, C₂-C₆ alkenyl, C₂-C₆ alkynyl, C₁-C₆ alkoxyl, C₁-C₆ thioalkyl, C(O)OC₁-C₆ alkyl, C(O)NH₂, SO₂C₁-C₆ alkyl, SO₂C₆-C₁₀ aryl, SO₂NH₂, C(O)NH(C₁-C₆ alkyl), C(O)N(C₁-C₆ alkyl)₂, SO₂NH(C₁-C₆ alkyl), SO₂N(C₁-C₆ alkyl)₂, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl; or

R₇ and R₈, together with the carbon atoms to which they are attached, form phenyl or a 5- or 6-membered heteroaryl having 1 to 3 heteroatoms.

The compounds of any of Formulae (I), (Ia), (Ib), and (Ic) can generally include one or more of the following features, when applicable.

For example, ring A is optionally substituted C₄-C₅ cycloalkyl.

For example, ring A is unsubstituted C₃-C₆ cycloalkyl, e.g. unsubstituted cyclobutyl or unsubstituted cyclopentyl.

For example, ring A is C₃-C₆ cycloalkyl substituted with one or more C₁-C₃ alkyl, e.g., with one C₁-C₃ alkyl. For compounds of Formula (I), (Ia), (Ib) and (Ic), ring A is substituted or unsubstituted cyclobutyl in some embodiments.

In some compounds of Formula (Ic), ring A may be unsubstituted cyclopentyl.

For example, one of R₁ and R₂ is H and the other is C₁-C₆ alkyl optionally substituted with halo or is C₃-C₈ cycloalkyl optionally substituted with C₁-C₆ alkyl. Thus, in some embodiments, including compounds of Formula (Ia), one of R₁ and R₂ may be H and the other may be isopropyl or t-butyl. Said isopropyl or t-butyl may be unsubstituted or may be substituted with one or more halo groups, e.g. 1-fluoro-prop-2-yl.

For example, one of R₁ and R₂ is C₁-C₆ alkyl optionally substituted with halo and the other is C₁-C₆ alkyl optionally substituted with halo or is C₂-C₆ alkynyl or C₃-C₈ cycloalkyl optionally substituted with C₁-C₆ alkyl.

Generally, R₁ and R₂, together with the nitrogen atom to which they are attached, may form an optionally substituted 4 to 12-membered saturated heterocycloalkyl ring having 0 to 2 additional heteroatoms (e.g., azetidinyl, pyrrolidinyl, imidazolidinyl, pyrazolidinyl, oxazolidinyl, isoxazolidinyl, triazolidinyl, piperidinyl, piperazinyl, 1,4-diazepanyl, 1,4-oxazepanyl, morpholinyl, 3-azabicyclo[3.2.1]octan-3-yl, 2-azabicyclo[2.2.1]heptan-2-yl, and 2-oxa-5-azabicyclo[2.2.1]heptan-5-yl, and azepanyl).

For example, R₁ and R₂, together with the nitrogen atom to which they are attached, form an optionally substituted 5 to 8-membered saturated heterocycloalkyl ring having 0 to 2 additional heteroatoms.

For example, the 5 to 8-membered saturated heterocycloalkyl ring formed by R₁ and R₂ is unsubstituted.

For example, the 5 to 8-membered saturated heterocycloalkyl ring formed by R₁ and R₂ is substituted with 1-3 substituents selected from the group consisting of halo, cyano, C₁-C₆ alkyl, C₁-C₆ haloalkyl, and C₁-C₆ alkoxyl.

For example, the 5 to 8-membered saturated heterocycloalkyl ring formed by R₁ and R₂ is substituted with one or two substituents selected from the group consisting of fluoro, cyano, CH₃, CH₂CH₃, CF₃, and OCH₃.

For example, the 5 to 8-membered saturated heterocycloalkyl ring formed by R₁ and R₂ is selected from piperidin-1-yl, pyrrolidin-1-yl, azepane-1-yl, morpholin-4-yl, 3-azabicyclo[3.2.1]octan-3-yl, 2-azabicyclo[2.2.1]heptan-2-yl, and 2-oxa-5-azabicyclo[2.2.1]heptan-5-yl.

In some embodiments, including compounds of Formulae (Ia), (Ib) and (Ic), the 5 to 8-membered saturated heterocycloalkyl ring formed by R₁ and R₂ may suitably be piperidin-1-yl. Said piperidinyl may be unsubstituted. Alternatively, said piperidinyl may be substituted with a single residue selected from halo (typically fluoro) or cyano. Typically said piperidinyl may be substituted at the 3-position.

Alternatively, the 5 to 8-membered saturated heterocycloalkyl ring formed by R₁ and R₂ is selected from morpholin-4-yl, azepane-1-yl, 3-azabicyclo[3.2.1]octan-3-yl, 2-azabicyclo[2.2.1]heptan-2-yl, and 2-oxa-5-azabicyclo[2.2.1]heptan-5-yl.

For example in some embodiments, including compounds of Formula (Ia), the 5 to 8-membered saturated heterocycloalkyl ring formed by R₁ and R₂ may suitably be 2-oxa-5-azabicyclo[2.2.1]heptan-5-yl, which is preferably unsubstituted.

For example, the 5 to 8-membered saturated heterocycloalkyl ring formed by R₁ and R₂, when substituted with one or more fluoro, is not substituted at the 2-position with fluoro (e.g., the ring is a 6-membered ring that is substituted at the 3-, 4-, or both positions, with fluoro).

For example, the 5 to 8-membered saturated heterocycloalkyl ring formed by R₁ and R₂, when substituted with one or more C₁-C₆ alkoxyl, is not substituted at the 2-position with C₁-C₆ alkoxyl (e.g., the ring is a 6-membered ring that is substituted at the 3-, 4-, or both positions, with C₁-C₆ alkoxyl).

For example, the 5 to 8-membered saturated heterocycloalkyl ring formed by R₁ and R₂, when substituted with one or more cyano, is not substituted at the 2-position with cyano (e.g., the ring is a 6-membered ring that is substituted at the 3-, 4-, or both positions, with cyano).

As mentioned above, R₃ and R₄ may be both H, but in other embodiments, one of R₃ and R₄ may be H and the other may be C₁-C₆ alkyl, C₂-C₆ alkenyl, or C₂-C₆ alkynyl, for example.

Alternatively, R₃ and R₄, together with the carbon atom to which they are attached, may form C₃-C₆ cycloalkyl.

Alternatively, in some embodiments, including compounds of Formula (Ia), R₅ may be OR_{S2}, in which R_{S2} is H or C₁-C₆ alkyl.

In some embodiments it is preferred that R_{S2} is H. For example, for some compounds of Formula (Ia) R_{S2} is preferably H.

Generally, R₆ may be H.

In some compounds of Formula (Ia) for example, R₆ is preferably H.

Similarly for the compounds of Formula (Ib), R₆ may be H in some embodiments when p is 1.

Alternatively, in some embodiments, R₆ may be C₁-C₆ alkyl (e.g., methyl) for example.

In a further alternative, R₅ and R₆, together with the carbon atom to which they are attached, may form C═O, as in the compounds of Formula (Ic) for example.

For example, each of R₇ and R₈, independently, is H, halo, or cyano.

For example, at least one of R₇ and R₈ is halo.

For example, each of R₇ and R₈ is chloro.

For example, each of R₇ and R₈ is not H.

For example, R₇ and R₈, together with the carbon atoms to which they are attached, form phenyl or a 5- or 6-membered heteroaryl having 1 to 3 heteroatoms.

For example, R₇ and R₈, together with the carbon atoms to which they are attached, form phenyl.

For example, R₇ and R₈, together with the carbon atoms to which they are attached, form a 5- or 6-membered heteroaryl having 1 to 3 heteroatoms, e.g., pyridyl, pyrrolyl, furanyl, thienyl, thiazolyl, oxazolyl, imidazolyl, pyrazolyl, isoxazolyl, triazolyl, oxadiazolyl, pyridazinyl, pyrazinyl, and pyrimidyl.

For example, R₉ is H.

For example, R₁₀ is H.

For example, R₁₁ is H.

Representative compounds of the present disclosure include compounds listed in Table 1.

TABLE 1

| Compound | |
|---|---|
| No. | Structure |
| 1 | 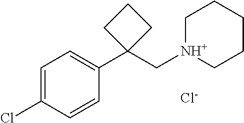 [SMILES: Clc1ccc(C2(C[NH+]3CCCCC3)CCC2)cc1 ; [Cl-]] |
| 2 | 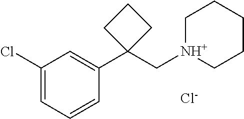 [SMILES: Clc1cccc(C2(C[NH+]3CCCCC3)CCC2)c1 ; [Cl-]] |
| 3 | 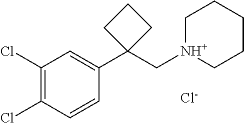 [SMILES: Clc1ccc(C2(C[NH+]3CCCCC3)CCC2)cc1Cl ; [Cl-]] |
| 4 | 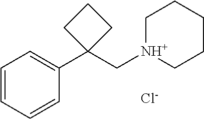 [SMILES: c1ccc(C2(C[NH+]3CCCCC3)CCC2)cc1 ; [Cl-]] |
| 5 | 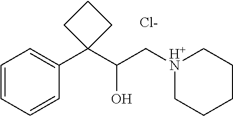 |
| 6 | 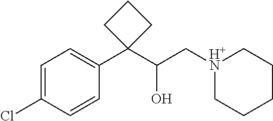 |
| 7 | 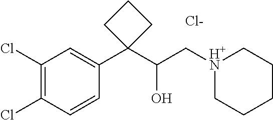 |
| 8 | 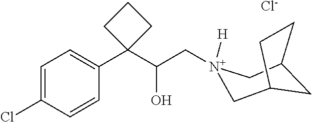 [SMILES: OC(C[NH+]1CC2CCC(C2)C1)C1(c2ccc(Cl)cc2)CCC1 ; [Cl-]] |
| 9 | 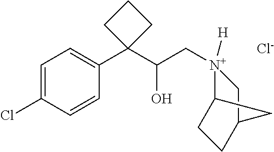 [SMILES: OC(C[NH+]1CC2CCC1C2)C1(c2ccc(Cl)cc2)CCC1 ; [Cl-]] |
| 10 | 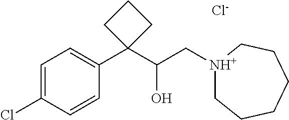 [SMILES: OC(C[NH+]1CCCCCC1)C1(c2ccc(Cl)cc2)CCC1 ; [Cl-]] |
| 11 | 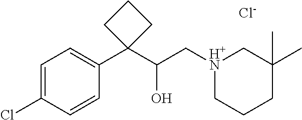 |
| 12 | 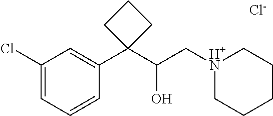 |
| 13 | 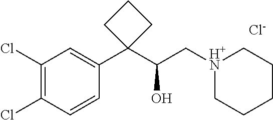 |
| 14 | 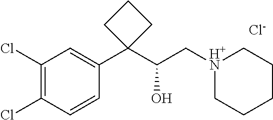 |
| 15 | 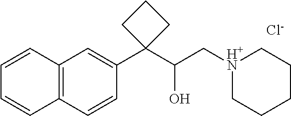 |
| 16 | 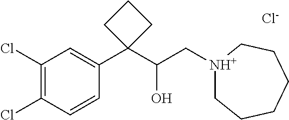 [SMILES: OC(C[NH+]1CCCCCC1)C1(c2ccc(Cl)c(Cl)c2)CCC1 ; [Cl-]] |
| 17 | 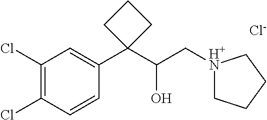 |
| 18 | 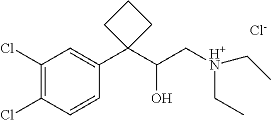 |
| 19 | 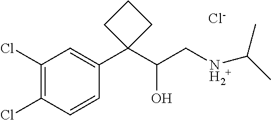 [SMILES: CC(C)[NH2+]CC(O)C1(c2ccc(Cl)c(Cl)c2)CCC1 ; [Cl-]] |
| 20 | 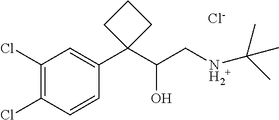 [SMILES: CC(C)(C)[NH2+]CC(O)C1(c2ccc(Cl)c(Cl)c2)CCC1 ; [Cl-]] |
| 21 | 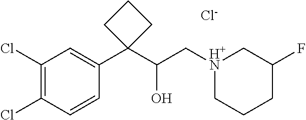 |
| 22 | 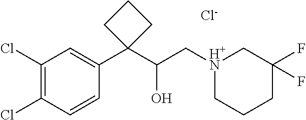 |
| 23 | 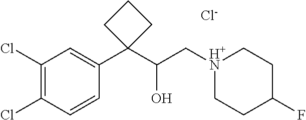 |
| 24 | 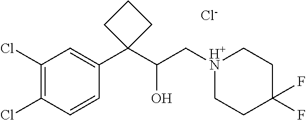 |
| 25 | 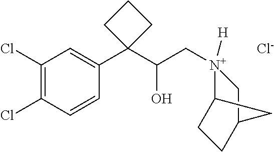 [SMILES: OC(C[NH+]1CC2CCC1C2)C1(c2ccc(Cl)c(Cl)c2)CCC1 ; [Cl-]] |
| 27 | 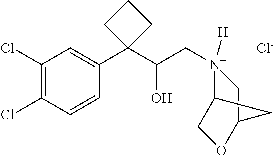 [SMILES: OC(C[NH+]1CC2CC1CO2)C1(c2ccc(Cl)c(Cl)c2)CCC1 ; [Cl-]] |
| 29 | 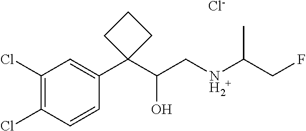 [SMILES: [Cl-] ; CC(CF)[NH2+]CC(O)C1(c2ccc(Cl)c(Cl)c2)CCC1] |
| 30 | 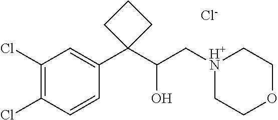 |
| 31 | 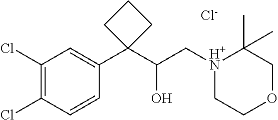 |
| 32 | 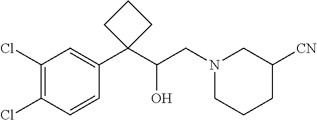 [SMILES: N#CC1CCCN(CC(O)C2(c3ccc(Cl)c(Cl)c3)CCC2)C1] |
| 33 | 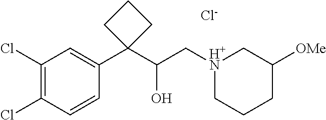 |
| 34 | 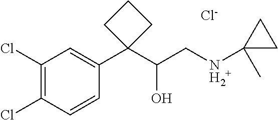 [SMILES: CC1([NH2+]CC(O)C2(c3ccc(Cl)c(Cl)c3)CCC2)CC1 ; [Cl-]] |
| 35 | 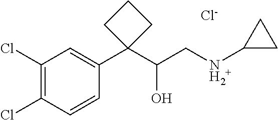 [SMILES: OC(C[NH2+]C1CC1)C1(c2ccc(Cl)c(Cl)c2)CCC1 ; [Cl-]] |
| 36 | 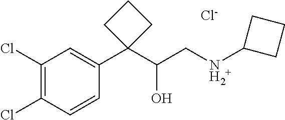 [SMILES: OC(C[NH2+]C1CCC1)C1(c2ccc(Cl)c(Cl)c2)CCC1 ; [Cl-]] |
| 38 | 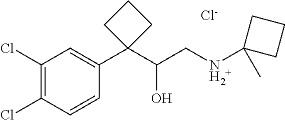 [SMILES: CC1([NH2+]CC(O)C2(c3ccc(Cl)c(Cl)c3)CCC2)CCC1 ; [Cl-]] |
| 39 | 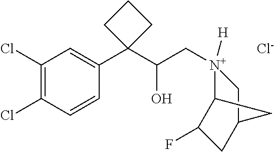 [SMILES: OC(C[NH+]1CC2CC(F)C1C2)C1(c2ccc(Cl)c(Cl)c2)CCC1 ; [Cl-]] |
| 42 | 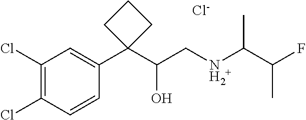 [SMILES: CC(F)C(C)[NH2+]CC(O)C1(c2ccc(Cl)c(Cl)c2)CCC1 ; [Cl-]] |
| 47 | 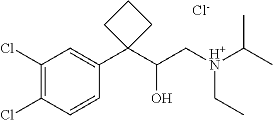 |
| 48 | 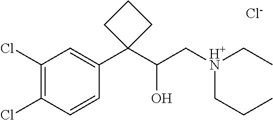 |
| 59 | 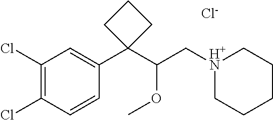 |
| 60 | 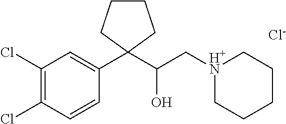 |
| 61 | 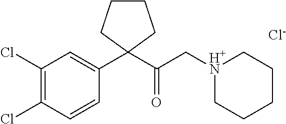 |
| 66 | 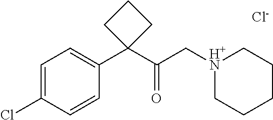 |
| 67 | 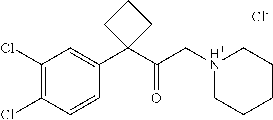 |
| 68 | 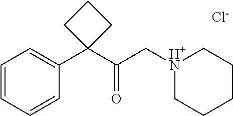 |
| 69 | 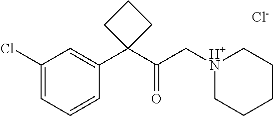 |
| 71 | 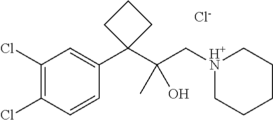 |

In certain embodiments, this disclosure also relates to a compound of Formula (II) or a pharmaceutically acceptable salt thereof:

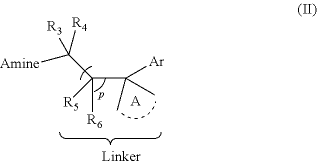

In the formula above, “Amine” is optionally substituted cyclic or acyclic amine (such as pyrrolidine, piperidine, morpholine, dialkylamine, any of the moieties in the compounds listed in Table 1 that correspond to —NR₁R₂ in Formula (I), and other primary, secondary, tertiary, or quaternary amines); “Linker” is —(CR₃R₄)—(CR₅R₆)ₚ-cycloalkyl ring A-, in which R₃, R₄, R₅, R₆ and ring A as defined herein, e.g., for Formula (I), (Ia), (Ib), or (Ic) and corresponding moieties in the compounds listed in Table 1; and “Ar” is optionally substituted aryl or heteroaryl, such as phenyl, naphthyl, pyridyl, pyrimidyl, benzoimidazolyl, and any of the moieties in the compounds listed in Table 1 that correspond to

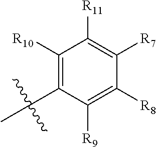

### in Formula (I).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated hydrocarbon mono- or multi-ring (e.g., fused, bridged, or spiro rings) system having 3 to 30 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, and adamantyl. The term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

The term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl (benzyl)). An “alkylaryl” moiety is an aryl substituted with an alkyl (e.g., methylphenyl).

As used herein, “alkyl linker” is intended to include C₁, C₂, C₃, C₄, C₅ or C₆ straight chain (linear) saturated divalent aliphatic hydrocarbon groups and C₃, C₄, C₅ or C₆ branched saturated aliphatic hydrocarbon groups. For example, C₁-C₆ alkyl linker is intended to include C₁, C₂, C₃, C₄, C₅ and C₆ alkyl linker groups. Examples of alkyl linker include, moieties having from one to six carbon atoms, such as, but not limited to, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂—), n-pentyl (—CH₂CH₂CH₂CH₂CH₂—), s-pentyl (—CHCH₃CH₂CH₂CH₂—) or n-hexyl (—CH₂CH₂CH₂CH₂CH₂CH₂—).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)_{q}, where q=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

As used herein, “carbocycle” or “carbocyclic ring” is intended to include any stable monocyclic, bicyclic or tricyclic ring having the specified number of carbons, any of which may be saturated, unsaturated, or aromatic. Carbocycle includes cycloalkyl and aryl. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” or “heterocyclic group” includes any ring structure (saturated, unsaturated, or aromatic) which contains at least one ring heteroatom (e.g., N, O or S). Heterocycle includes heterocycloalkyl and heteroaryl. Examples of heterocycles include, but are not limited to, morpholine, pyrrolidine, tetrahydrothiophene, piperidine, piperazine, oxetane, pyran, tetrahydropyran, azetidine, and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

The term “hydroxy” or “hydroxyl” includes —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” includes compounds and moieties which contain a carbon connected with a double bond to an oxygen atom (written as C═O or C(O)). Examples of moieties containing a carbonyl include, but are not limited to, aldehydes, ketones, carboxylic acids, amides, esters, anhydrides, etc.

The term “carboxyl” refers to —C(O)OH or its C₁-C₆ alkyl ester.

“Acyl” includes moieties that contain the acyl radical (RC(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen bonded to two carbon atoms or heteroatoms. For example, the term includes “alkoxyalkyl,” which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls, and alkthioalkynyls. The term “alkthioalkyls” include moieties with an alkyl, alkenyl, or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” refers to unsubstituted or substituted —NH₂. “Alkylamino” includes groups of compounds wherein nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

Compounds of the present disclosure that contain nitrogens can be converted to N-oxides by treatment with an oxidizing agent (e.g., 3-chloroperoxybenzoic acid (mCPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Calm et al., Angew. Chem. 1966, 78, 413; Calm and Ingold, J. Chem. Soc. 1951 (London), 612; Calm et al., Experientia 1956, 12, 81; Calm, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

Furthermore, the structures and other compounds discussed in this disclosure include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

It is to be understood that the compounds of the present disclosure may be depicted as different tautomers. It should also be understood, that when compounds have tautomeric forms, all tautomeric forms are intended to be included in the scope of the present disclosure, and the naming of the compounds does not exclude any tautomer form. It will be understood that certain tautomers may have a higher level of activity than others.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of any of Formulae disclosed herein include the compounds themselves, as well as their salts, their esters, their solvates, and their prodrugs, if applicable. A salt, for example, can be formed between an anion and a positively charged group (e.g., quaternary amino) on a cycloalkyl amine compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). Suitably the compounds of the disclosure may be provided and administered in the form of their hydrochloride salts. The term “pharmaceutically acceptable anion” refers to an anion suitable for forming a pharmaceutically acceptable salt. Likewise, a salt can also be formed between a cation and a negatively charged group (e.g., carboxylate) on a cycloalkyl amine compound. Suitable cations include sodium ion, potassium ion, magnesium ion, calcium ion, and an ammonium cation such as tetramethylammonium ion. The cycloalkyl amine compounds also include those salts containing quaternary nitrogen atoms. Examples of prodrugs include esters and other pharmaceutically acceptable derivatives, which, upon administration to a subject, are capable of providing active cycloalkyl amine compounds.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are cycloalkyl amine compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### 2. SYNTHESIS OF CYCLOALKYL AMINE COMPOUNDS

The present disclosure provides methods for the synthesis of the compounds of any Formula disclosed herein. The present disclosure also provides detailed methods for the synthesis of various disclosed compounds of the present disclosure according to the following schemes as shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the disclosure remains operable. Moreover, two or more steps or actions can be conducted simultaneously.

The synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester, or prodrug thereof.

Compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

Compounds of the present disclosure can be conveniently prepared by a variety of methods familiar to those skilled in the art. The compounds of this disclosure with any Formula disclosed herein may be prepared according to the procedures illustrated in Schemes 1 and 2 below, from commercially available starting materials or starting materials which can be prepared using literature procedures. The A and R groups (such as R₁, R₂, R₄, R₅, R₆, R₇, and R₈) in Schemes 1-2 are as defined in any of Formulae disclosed herein, unless otherwise specified.

One of ordinary skill in the art will note that, during the reaction sequences and synthetic schemes described herein, the order of certain steps may be changed, such as the introduction and removal of protecting groups.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

Preferred protecting groups include, but are not limited to:

For a hydroxyl moiety: TBS, benzyl, THP, Ac

For carboxylic acids: benzyl ester, methyl ester, ethyl ester, allyl ester

For amines: Cbz, BOC, DMB

For diols: Ac (×2) TBS (×2), or when taken together acetonides

For thiols: Ac

For benzimidazoles: SEM, benzyl, PMB, DMB

For aldehydes: di-alkyl acetals such as dimethoxy acetal or diethyl acetyl.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

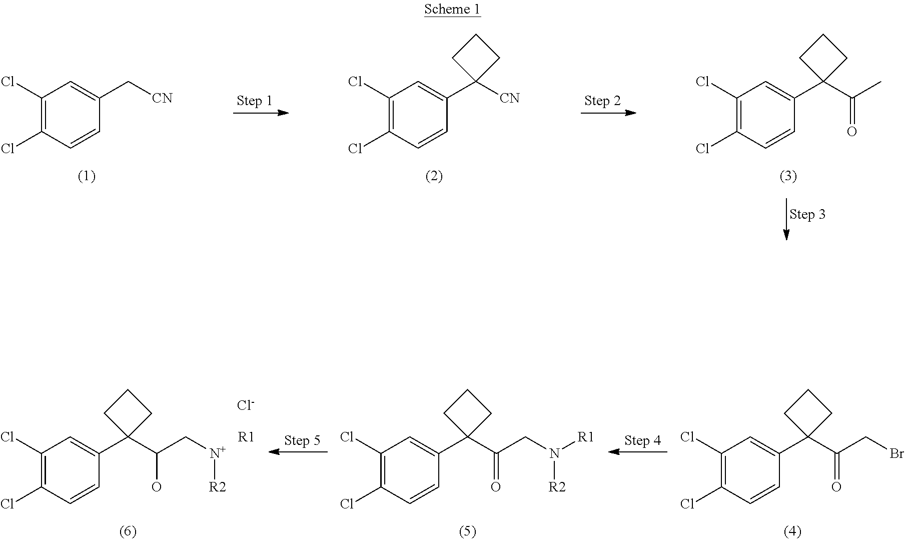

Scheme 1 shows the syntheses of two discrete series: (i) compounds containing an oxo-ethyl linker, i.e., the “oxo-ethyl linker series” such as structure (5) in the scheme above; (ii) compounds containing a hydroxy-ethyl linker, i.e., the “hydroxy-ethyl linker series” such as structure (6) shown above.

More specifically, a phenylacetonitrile such as 3,4-dichlorophenylacetonitrile (1) is treated with, e.g., sodium hydride and 1,3-dibromopropane at, e.g., ambient temperature, which furnishes the cyclobutylnitrile (2) (Step 1). The nitrile (2) is treated with, e.g., methyl magnesium bromide at an elevated temperature, e.g., 75° C., which after aqueous acid treatment furnishes the methyl ketone (3) (Step 2). Reaction of the methyl ketone (3) with, e.g., bromine and hydrobromic acid at, e.g., 0° C., provides the bromomethyl ketone (4) (Step 3). The bromomethyl ketone (4) is then reacted with an amine in the presence of e.g., either excess amine or potassium carbonate, to provide the intended amino-ketone (5) of the oxo-ethyl linker series (Step 4). The amino-ketones (5) are then treated with, e.g., sodium borohydride at, e.g., 0° C. to provide the hydroxy amines, which are treated with, e.g., 4M HCl in dioxan, to provide the intended hydrochloride salts (6) of the hydroxy-ethyl linker series (Step 5).

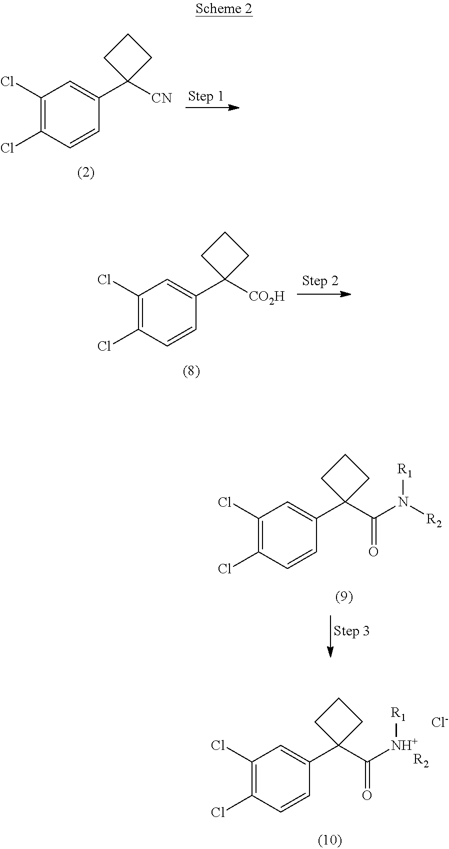

Scheme 2 above shows the synthesis of the series of compounds that include a methyl linker, i.e., the “methyl linker series” such as structure (10) above. In particular, the nitrile (2) is heated at an elevated temperature (e.g., 190° C.) with potassium hydroxide in diethylene glycol, which provides the acid (8) after aqueous acid treatment (Step 1). The acid (8) is then treated with an amine in the presence of the coupling agent, e.g., HATU and an amine base at, e.g., 0° C. to ambient temperature, giving the amides (9) (Step 2). The amides (9) are then treated with, e.g., lithium aluminum hydride at, e.g., 0° C., to provide the amines, which are then treated with, e.g., 4M HCl in dioxan, to provide the intended hydrochloride salts (10) of the methyl linker series (Step3).

### 3. METHODS OF TREATMENT

Compounds of the present disclosure inhibit neurotransmitter reuptake, in particular, block the reuptake of dopamine and norepinephrine into presynaptic cells. This inhibition of neurotransmitter reuptake can increase the amount of neurotransmitter present in the synapse, thus helping to normalize the transmission of neuronal signals. Such normalization of neurotransmitter levels, particularly within the prefrontal cortex, may be useful in the treatment of CNS disorders. Accordingly, in one aspect of the disclosure, certain compounds disclosed herein are candidates for treating, or preventing CNS conditions and diseases. The method includes administering to a subject in need of such treatment, a therapeutically effective amount of a compound of the present disclosure, or a pharmaceutically acceptable salt, prodrug, metabolite, polymorph, solvate, or stereoisomeror thereof.

As used herein, a “subject in need thereof” is a subject having a CNS disorder in which, e.g., an imbalance of neurotransmitters in the brain plays a part, or a subject having an increased risk of developing such disorder relative to the population at large. Preferably, a subject in need thereof has a CNS disorder that is caused by or associated with an abnormally insufficient amount of neurotransmitters. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

As used herein, the term “CNS disorder” refers to a disease that can affect either the spinal cord or brain, both of which are part of the central nervous system. A CNS disorder associated with an imbalance of neurotransmitters in the brain can be caused by, e.g., trauma, infections, neurodegeneration, tumors, autoimmune disorders, stroke, and genetic predisposition.

Exemplary CNS conditions or disorders that may be treated using one or more compounds of the present disclosure include, but are not limited to, movement disorders, depressive disorders, sleep disorders (e.g., narcolepsy, excessive daytime sleepiness such as excessive daytime sleepiness in patients with Parkinson's Disease or Multiple Sclerosis, other hypersomnias such as primary or idiopathic hypersomnia, Kleine-Levin Syndrome, Shift-work Sleep Disorder, Circadian Rhythm Disorder, REM Behavioral Disorder), apathy as a component of neurological, psychiatric or neurodegenerative disorders, obesity, sexual dysfunction (e.g., iatrogenic sexual dysfunction), substance abuse such as alcohol or cocaine abuse and nicotine dependence, and cognitive dysfunction such as attention deficit disorder (ADD), attention deficit hyperactivity disorder (ADHD), Lewy Body Disease, Amyotrophic Lateral Sclerosis (ALS), executive dysfunction as a component of Parkinson's Disease, affective sequelae of traumatic brain injury, neuropsychological sequelae of traumatic brain injury, cognitive late effects secondary to CNS chemotherapy, neurocognitive dysfunction following coronary artery bypass surgery or acute stroke cognitive sequelae and cognitive impairment associated with pre-manifest, early-stage and late-stage Huntington's Disease or Multiple Sclerosis, and fronto-temporal dementia such as Alzheimer's Disease.

As used herein, “contacting a cell” refers to a condition in which a compound or other composition of matter is in direct contact with a cell, or is close enough to induce a desired biological effect in a cell.

As used herein, “candidate compound” refers to a compound of the present disclosure, or a pharmaceutically acceptable salt, ester, prodrug, metabolite, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present disclosure, or a pharmaceutically acceptable salt, ester, prodrug, metabolite, polymorph or solvate thereof. The biological or medical response can be alleviation or elimination of one or more symptoms or complications of a CNS disorder. The biological response or effect can also include a change in dopamine (or serotonin or norepinephrine) uptake that occurs in vitro or in an animal model, as well as other biological changes that are observable in vitro or ex vivo. In vitro or in vivo biological assays can include, but are not limited to, functional in vitro cellular assays using recombinant human cell lines to detect inhibition of dopamine, norepinephrine or serotonin reuptake, in vitro radioligand binding assays using cell membrane preparations stably expressing human recombinant DAT, NET or SERT receptors; or in vivo microdialysis assays to quantify the extracellular levels of dopamine, norepinephrine, and serotonin neurotransmitters in the mammalian brain; such as the assays described in Owens et al., The Journal of Pharmacology and Experimental Therapeutics 283:1305-1322, 1997; Mason et al., The Journal of Pharmacology and Experimental Therapeutics 323:720-729, 2007; Eshleman et al., The Journal of Pharmacology and Experimental Therapeutics 289:877-885, 1999; Skolnick et al., European Journal of Pharmacology, 461(2-3):99-104, 2003; or Nirogi et al., Journal of Chromatography B, 913-914, p. 41-47, 2013; and the assays described herein.

Most drugs exert their effects in defined target tissues into which drugs have to distribute from a central compartment. Direct assessment of drug effects on tissue biochemistry is a rational way to provide clinically meaningful evidence of in vivo efficacy (Berridge et al, Biol Psychiatry 69, e101-e111, 2011; Madras et al, Biol Psychiatry 57, 1397-1409, 2005). Microdialysis is a minimally-invasive sampling technique that can be used to continuously measure the concentration of free, unbound analyte concentrations in the extracellular fluid of target tissue (Kehr, Modern techniques in Neuroscience Research, U. Windhorst and H. Johansson, Eds., Springer Verlag, 1991). Brain microdialysis can therefore be employed to show how the compounds described herein affect the extracellular levels of dopamine, norepinephrine, and serotonin neurotransmitters in the rat brain. Typically, compounds that increase dopamine or norepinephrine level or both by 75 (seventy five) percent or more in the striatum, nuculeus accumbens, and especially the prefrontal cortex, relative to baseline neurotransmitter levels in untreated subject such as an animal, are suitable candidates for treating or preventing CNS disorders or conditions.

As used herein, “monotherapy” refers to the administration of a single active or therapeutic compound to a subject in need thereof. Preferably, monotherapy will involve administration of a therapeutically effective amount of an active compound. For example, monotherapy with one of the compound of the present disclosure, or a pharmaceutically acceptable salt, prodrug, metabolite, analog or derivative thereof, to a subject in need of treatment of a CNS disorder. Monotherapy may be contrasted with combination therapy, in which a combination of multiple active compounds is administered, preferably with each component of the combination present in a therapeutically effective amount. In one aspect, monotherapy with a compound of the present disclosure, or a pharmaceutically acceptable salt, prodrug, metabolite, polymorph or solvate thereof, is more effective than combination therapy in inducing a desired biological effect.

As used herein, “treating” or “treat” describes the management and care of a patient for the purpose of combating a disease, condition, or disorder and includes the administration of a compound of the present disclosure, or a pharmaceutically acceptable salt, prodrug, metabolite, polymorph or solvate thereof, to alleviate the symptoms or complications of a disease, condition or disorder, or to eliminate the disease, condition or disorder. The term “treat” can also include treatment of a cell in vitro or an animal model.

A compound of the present disclosure, or a pharmaceutically acceptable salt, prodrug, metabolite, polymorph or solvate thereof, can also be used to prevent a disease, condition or disorder, or used to identify suitable candidates for such purposes. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

A compound of the present disclosure, or a pharmaceutically acceptable salt, prodrug, metabolite, polymorph or solvate thereof, can modulate the activity of a molecular target (e.g., a dopamine receptor). Modulating refers to stimulating or inhibiting an activity of a molecular target. Preferably, a compound of the present disclosure, or a pharmaceutically acceptable salt, prodrug, metabolite, polymorph or solvate thereof, modulates the activity of a molecular target if it stimulates or inhibits the activity of the molecular target by at least 2-fold relative to the activity of the molecular target under the same conditions but lacking only the presence of said compound. More preferably, a compound of the present disclosure, or a pharmaceutically acceptable salt, prodrug, metabolite, polymorph or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo.

A compound of the present disclosure, or a pharmaceutically acceptable salt, prodrug, metabolite, polymorph or solvate thereof, does not significantly modulate the activity of a molecular target if the addition of the compound does not stimulate or inhibit the activity of the molecular target by greater than 10% relative to the activity of the molecular target under the same conditions but lacking only the presence of said compound.

As used herein, “combination therapy” or “co-therapy” includes the administration of a compound of the present disclosure, or a pharmaceutically acceptable salt, prodrug, metabolite, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations of the present disclosure.

“Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the therapeutic agents as described above in further combination with other biologically active ingredients and non-drug therapies (e.g., surgery, speech therapy, or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

### 4. PHARMACEUTICAL COMPOSITIONS

The present disclosure also provides pharmaceutical compositions comprising a compound of any Formula disclosed herein in combination with at least one pharmaceutically acceptable excipient or carrier.

A “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is a CNS disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. As used herein, the term “dosage effective manner” refers to amount of an active compound to produce the desired biological effect in a subject or cell.

The pharmaceutical compositions can be included in a container, pack, or dispenser together with instructions for administration or use.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

The compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds of the present disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p 1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described herein.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples below are for purposes of illustration and not limitation of the claims that follow.

### 5. EXAMPLES

### Example 1

### Synthesis of 1-[(1-phenylcyclobutyl)methyl]piperidin-1-ium chloride (Compound 4)

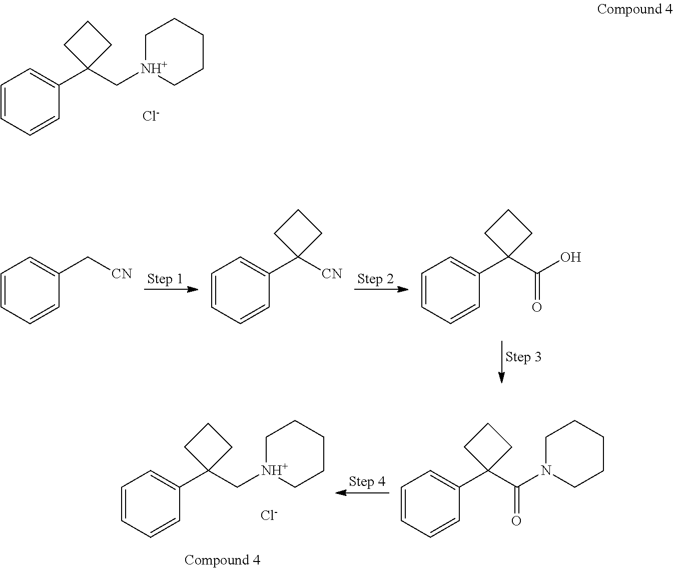

### Step 1: 1-Phenylcyclobutane-1-carbonitrile

To a stirred slurry of 60% NaH (7.5 g, 188.03 mmol) in DMSO (25 mL) was added a mixture of phenyl acetonitrile (10 g, 85.47 mmol) and 1,3-dibromo propane (18.9 g, 94.01 mmol) in Et₂O (30 mL) drop-wise. The reaction was stirred at room temperature for 18 h, cooled to 0° C. and quenched with 2-propanol (25 mL), then diluted with water (25 mL). The aqueous layer was extracted with hexane (2×100 mL) and the combined organic layer was dried over Na₂SO₄ and concentrated under reduced pressure to afford the required crude compound, which was purified by column chromatography (silica gel, eluting with 5% ethyl acetate/hexane) to obtain the title compound (5.6 g, 41% yield) as a colorless viscous liquid. ¹H NMR (300 MHz, CDCl₃) δ 7.49-7.34 (m, 5H), 2.83 (tdd, J=8.9, 4.5, 2.6 Hz, 2H), 2.72-2.52 (m, 2H), 2.52-2.30 (m, 1H), 2.08 (dtt, J=11.5, 9.1, 4.4 Hz, 1H).

### Step 2: 1-(Phenyl)cyclobutane-1-carboxylic acid

To a stirred solution of 1-phenylcyclobutanecarbonitrile (1.6 g, 10.19 mmol) in diethylene glycol (10 mL) was added KOH (5.7 g, 101.9 mmol). The reaction was heated to reflux (190° C.) for 16 h, then cooled to 0° C. and poured onto crushed ice (50 g). The aqueous layer was washed with Et₂O (2×50 mL) and acidified slowly with 6N HCl maintaining the temperature below 25° C. The solid was filtered and washed with water (50 mL) and dried under vacuum to yield the title compound (1.0 g, 55%) as an off white solid.

### Step 3: 1-[1-(Phenyl)cyclobutanecarbonyl]piperidine

To a stirred solution of 1-(phenyl)cyclobutane-1-carboxylic acid (600 mg, 3.46 mmol) and HATU (2 g, 5.19 mmol) in THF was added di-isopropylethylamine (1.3 g, 10.38 mmol). The reaction mixture was stirred at 0° C. for 1 h, then piperidine (880 mg, 10.38 mmol) added at 0° C. After stirring at room temperature for 16 h the reaction mixture was quenched with water (10 mL) and extracted with EtOAc (2×10 mL). The combined organic layer was dried over Na₂SO₄, concentrated to dryness and purified by silica gel column chromatography (5-10% EtOAc/hexane) to afford the title compound (600 mg, 73%) as a pale yellow viscous liquid.

### Step 4: 1-[(1-Phenylcyclobutyl)methyl]piperidin-1-ium chloride (Compound 4)

To a stirred solution of 1-[1-(phenyl)cyclobutanecarbonyl]piperidine (600 mg, 2.46 mmol) in THF was added LAH (1M in THF; 3.4 mL, 3.70 mmol) drop-wise at 0° C. After 3 h, the reaction mixture was quenched with saturated Na₂SO₄ solution (5 mL) and extracted with EtOAc (2×25 mL). The combined organic layer was dried over Na₂SO₄, concentrated to dryness and purified by silica gel column (10-15% EtOAc/hexane) to afford 1-[(1-phenylcyclobutyl)methyl]piperidine (200 mg, 35%) as a colorless liquid. LC-MS: m/z=230.2 [M+H]⁺. To a stirred solution of 1-[(1-phenylcyclobutyl)methyl]piperidine (200 mg, 0.87 mmol) in 1,4-dioxane (2 mL) was added 4M HCl in dioxane (2 mL) at 0° C. and the reaction stirred at room temperature for 16 h. The reaction mixture was concentrated to dryness and washed with Et₂O (10 mL), EtOAc (10 mL). The organic solvent was decanted and the resulting solid was dried under vacuum to yield the title compound (120 mg, 52% yield) as an off white solid. LC-MS: m/z=229.9 [M+H]⁺. ¹H NMR (400 MHz, DMSOd₆) δ 8.53 (bs, 1H), 7.41 (d, J=4.3 Hz, 4H), 7.30 (h, J=4.1 Hz, 1H), 3.60 (d, J=5.1 Hz, 2H), 2.99 (d, J=12.1 Hz, 2H), 2.76-2.63 (m, 2H), 2.44-2.29 (m, 4H), 2.07 (dq, J=11.2, 7.9 Hz, 1H), 1.78 (m, 1H), 1.57 (ddd, J=23.3, 10.2, 6.5 Hz, 5H), 1.27 (d, J=23.6 Hz, 1H).

### Example 2

### Synthesis of Compounds 1-3

The following compounds were synthesized using the same general synthetic protocols to those described in Example 1.

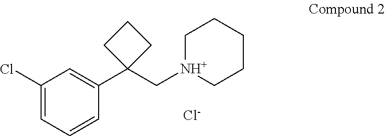 [SMILES: Clc1cccc(C2(C[NH+]3CCCCC3)CCC2)c1 ; [Cl-]]

### 1-{[1-(3-Chlorophenyl)cyclobutyl]methyl}piperidin-1-ium chloride

LC-MS: m/z=264.2 [M+H]⁺. ¹H NMR (400 MHz, DMSOd₆) δ 8.71 (bs, 1H), 7.50 (t, J=1.9 Hz, 1H), 7.47-7.38 (m, 2H), 7.36 (dt, J=7.3, 1.8 Hz, 1H), 3.62 (d, J=5.1 Hz, 2H), 3.04 (d, J=11.8 Hz, 2H), 2.73 (qd, J=14.4, 13.2, 7.3 Hz, 2H), 2.36 (t, J=7.7 Hz, 4H), 2.04 (dt, J=11.3, 7.9 Hz, 1H), 1.78 (dt, J=11.3, 7.6 Hz, 1H), 1.71-1.49 (m, 5H), 1.31 (dt, J=13.2, 8.1 Hz, 1H).

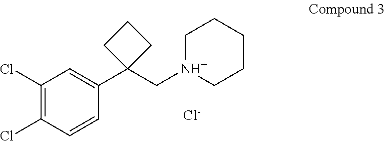 [SMILES: Clc1ccc(C2(C[NH+]3CCCCC3)CCC2)cc1Cl ; [Cl-]]

### 1-{[1-(3,4-Dichlorophenyl)cyclobutyl]methyl}piperidin-1-ium chloride

LC-MS: m/z=298.1 [M+H]⁺. ¹H NMR (300 MHz, DMSOd₆) δ 8.68 (bs, 1H), 7.71 (d, J=2.2 Hz, 1H), 7.66 (d, J=8.4 Hz, 1H), 7.45 (dd, J=8.4, 2.2 Hz, 1H), 3.63 (d, J=5.3 Hz, 2H), 3.09 (d, J=11.3 Hz, 2H), 2.87-2.68 (m, 2H), 2.36 (t, J=7.6 Hz, 4H), 2.10-1.92 (m, 1H), 1.85-1.48 (m, 6H), 1.31 (m, J=13.4 Hz, 1H).

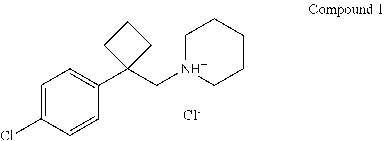 [SMILES: Clc1ccc(C2(C[NH+]3CCCCC3)CCC2)cc1 ; [Cl-]]

### 1-{[1-(4-Chlorophenyl)cyclobutyl]methyl}piperidin-1-ium chloride

LC-MS: m/z=234.2 [M+H]⁺. ¹H NMR (400 MHz, DMSOd₆) δ 8.90 (bs, 1H), 7.46 (s, 4H), 3.60 (d, J=5.1 Hz, 2H), 3.03 (d, J=12.0 Hz, 2H), 2.72 (dq, J=14.9, 8.0 Hz, 2H), 2.44-2.28 (m, 4H), 2.03 (qd, J=8.4, 2.3 Hz, 1H), 1.84-1.69 (m, 1H), 1.70-1.48 (m, 5H), 1.30 (dq, J=13.7, 7.3 Hz, 1H).

### Example 3

### Synthesis of 1-[2-Hydroxy-2-(1-phenylcyclobutyl)ethyl]piperidin-1-ium chloride

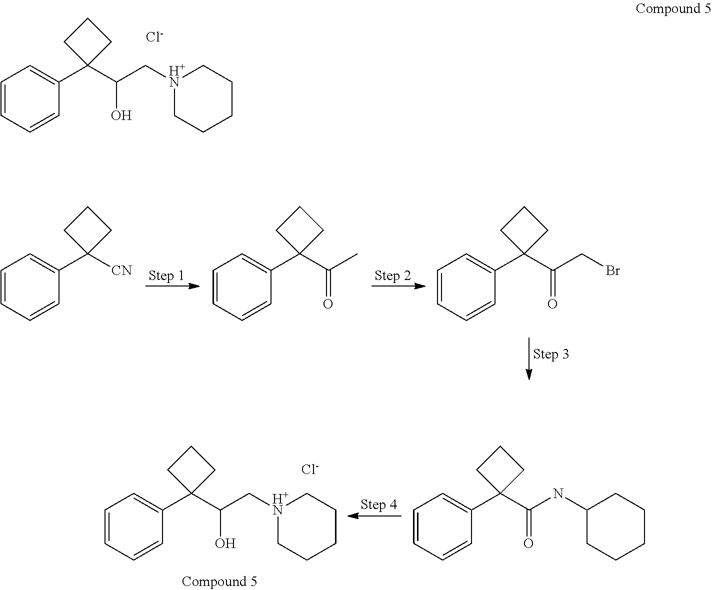

### Step 1: Synthesis of 1-(1-phenylcyclobutyl)ethan-1-one

To a stirred solution of 1-phenylcyclobutane-1-carbonitrile (4.0 g, 25.47 mmol) in toluene (25 mL) was slowly added methyl magnesium bromide (3M in Et₂O; 25.2 mL, 76.43 mmol) at 10° C. The reaction was warmed to 75° C. for 16 h then cooled to 0° C., poured onto crushed ice and quenched slowly with 6N HCl (20 mL). The resulting slurry was heated to 95° C. and stirred for 2 h, then cooled to room temperature and extracted with Et₂O (2×50 mL). The combined organic layers were washed with brine (25 mL), dried over Na₂SO₄ and concentrated under reduced pressure to afford the title compound (3.5 g, 79% yield) as a pale yellow viscous liquid. ¹H NMR (400 MHz, CDCl₃) δ 7.42-7.30 (m, 2H), 7.31-7.10 (m, 3H), 2.81-2.64 (m, 2H), 2.50-2.29 (m, 2H), 1.93 (s, 3H), 1.90-1.75 (m, 2H).

### Step 2: Synthesis of 2-bromo-1-(1-phenylcyclobutyl)ethan-1-one

To a stirred solution of 1-(1-phenylcyclobutyl)ethan-1-one (3.5 g, 20.11 mmol) in MeOH (25 mL) was added HBr (30% in AcOH; 0.22 mL, 0.80 mmol) and bromine (5.75 g, 36.19 mmol) at 0° C. After stirring at 0° C. for 16 h, the reaction mixture was carefully added to water (25 mL) over a period of 10 min while maintaining the reaction mixture temperature below 15° C. The resulting mixture was extracted with Et₂O (2×25 mL) and the combined ether layer washed with brine (25 mL), dried over Na₂SO₄ and concentrated under reduced pressure to give the title compound (3.5 g, crude) as a brown viscous liquid, which was used in the next step without further purification. ¹H NMR (300 MHz, CDCl₃) δ 7.46-7.16 (m, 5H), 3.86 (s, 2H), 2.93-2.68 (m, 2H), 2.61-2.40 (m, 2H), 2.11-1.80 (m, 2H).

### Step 3: Synthesis of 1-(1-phenylcyclobutyl)-2-(piperidin-1-yl)ethan-1-one

To a stirred solution of piperidine (1.4 g, 16.60 mmol) and potassium carbonate (9.5 g, 69.15 mmol) in acetone (20 mL) was added a slurry of 2-bromo-1-(1-phenylcyclobutyl)ethan-1-one (3.5 g, 13.83 mmol) and sodium iodide (2.47 g, 16.60 mmol) in acetone (10 mL). The reaction mixture was refluxed for 16 h, then cooled to ambient temperature and quenched with water (25 mL). The mixture was extracted with EtOAc (2×50 mL), the combined EtOAc layer was dried over Na₂SO₄, concentrated to dryness and purified by silica gel column (10-12% EtOAc/hexane) to afford the title compound (2.0 g, 57% yield) as colorless viscous liquid. LC-MS: m/z=258.2 [(M+H)]⁺.

### Step 4: Synthesis of 1-[2-hydroxy-2-(1-phenylcyclobutyl)ethyl]piperidin-1-ium chloride (Compound 5)

To a stirred solution of 1-(1-phenylcyclobutyl)-2-(piperidin-1-yl)ethan-1-one (500 mg, 1.94 mmol) in MeOH (20 mL) was added NaBH₄ (70 mg, 2.91 mmol) portionwise at 0° C. and the reaction stirred for 2 h. The reaction mixture was quenched with ice cold water over a period of 10 min, while maintaining the reaction mixture temperature below 15° C. MeOH was distilled from the reaction mixture at reduced pressure and the aqueous layer was extracted with CH₂Cl₂ (2×25 mL), dried over Na₂SO₄ and concentrated to dryness to obtain the crude product. Purification by silica gel chromatography (10-15% EtOAc/hexane) afforded 1-(1-phenylcyclobutyl)-2-(piperidin-1-yl)ethan-1-ol (350 mg, 70% yield) as a colorless viscous liquid. LC-MS: m/z=260.2 [(M+H)]⁺.

To a stirred solution of 1-(1-phenylcyclobutyl)-2-(piperidin-1-yl)ethan-1-ol (350 mg, 1.35 mmol) in 1,4-dioxane (2 mL) was added 4M HCl in dioxane (2 mL) at 0° C. and the mixture stirred at room temperature for 16 h. The mixture was concentrated to dryness and washed with Et₂O (10 mL) and EtOAc (10 mL). The organic solvent was decanted and the resulting solid was dried under vacuum to afford the title compound (100 mg, 25% yield) as an off-white solid. LC-MS: m/z=260.2 [M+H]⁺. ¹H NMR (400 MHz, DMSOd₆) δ 9.08 (bs, 1H), 7.32 (t, J=7.6 Hz, 2H), 7.24-7.11 (m, 3H), 5.91 (d, J=5.1 Hz, 1H), 4.27-4.16 (m, 1H), 3.44 (d, J=11.9 Hz, 1H), 3.32 (m, 1H), 2.96-2.64 (m, 3H), 2.50 (p, J=1.8 Hz, 1H), 2.37 (ddd, J=23.7, 10.2, 2.9 Hz, 2H), 2.22 (dq, J=19.2, 10.0, 9.3 Hz, 2H), 2.00 (dt, J=17.0, 8.6 Hz, 1H), 1.85-1.56 (m, 6H), 1.43-1.16 (m, 1H).

### Example 4

### Synthesis of Compounds 6-8, 10-12, 15-18, 21-25, 27, 30-36, 38-39, and 47-48

The following compounds were synthesized using the same general synthetic protocols to those described in Example 3.

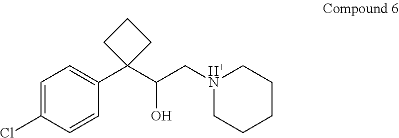

### 1-{2-[1-(4-Chlorophenyl)cyclobutyl]-2-hydroxyethyl}piperidin-1-ium chloride

LC-MS: m/z=294.2 [M+H]⁺. ¹H NMR (400 MHz, DMSOd₆) δ 9.23 (bs, 1H), 7.43-7.27 (d, 2H), 7.17 (d, J=2.3 Hz, 2H), 5.94 (d, J=5.1 Hz, 1H), 4.24 (dd, J=9.3, 5.0 Hz, 1H), 3.45 (d, J=12.0 Hz, 1H), 3.32 (m, 1H), 2.91 (dd, J=12.9, 6.6 Hz, 1H), 2.80 (tt, J=13.1, 9.1 Hz, 2H), 2.34 (ddd, J=16.6, 9.5, 3.3 Hz, 3H), 2.28-2.10 (m, 2H), 1.98 (q, J=9.0 Hz, 1H), 1.85-1.54 (m, 6H), 1.37-1.16 (m, 1H).

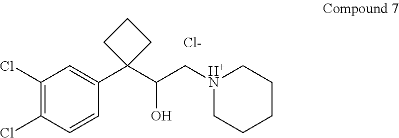

### 1-{2-[1-(3,4-Dichlorophenyl)cyclobutyl]-2-hydroxyethyl}piperidin-1-ium chloride

LC-MS: m/z=328.2 [M+H]⁺. ¹H NMR (400 MHz, DMSOd₆) δ 9.47 (bs, 1H), 7.57 (d, J=8.3 Hz, 1H), 7.37 (d, J=2.1 Hz, 1H), 7.15 (dd, J=8.3, 2.2 Hz, 1H), 5.99 (d, J=5.5 Hz, 1H), 4.31 (d, J=9.4 Hz, 1H), 3.43 (q, J=14.4 Hz, 3H), 3.06-2.96 (m, 1H), 2.85-2.74 (m, 2H), 2.40-2.24 (m, 3H), 2.14 (d, J=10.0 Hz, 1H), 2.06-1.95 (m, 1H), 1.90-1.58 (m, 6H), 1.40-1.19 (m, 1H).

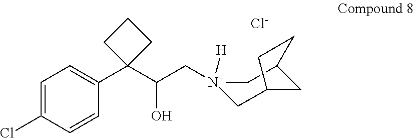 [SMILES: OC(C[NH+]1CC2CCC(C2)C1)C1(c2ccc(Cl)cc2)CCC1 ; [Cl-]]

### 3-{2-[1-(3-Chlorophenyl)cyclobutyl]-2-hydroxyethyl}-3-azabicyclo[3.2.1]octan-3-ium chloride

LC-MS: m/z=320.2 [M+H]⁺. ¹H NMR (400 MHz, DMSOd₆) δ 8.72 (bs, 1H), 7.43-7.24 (d, 2H), 7.19-7.06 (d, 2H), 5.80 (d, J=5.5 Hz, 1H), 4.28 (m, J=6.0 Hz, 1H), 3.32 (m, 2H), 2.96 (q, J=9.2, 6.8 Hz, 3H), 2.48-2.22 (m, 7H), 2.17-1.90 (m, 2H), 1.85-1.59 (m, 5H), 1.39 (dd, J=10.2, 5.0 Hz, 1H).

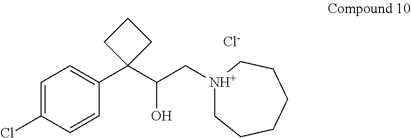 [SMILES: OC(C[NH+]1CCCCCC1)C1(c2ccc(Cl)cc2)CCC1 ; [Cl-]]

### 1-{2-[1-(3-Chlorophenyl)cyclobutyl]-2-hydroxyethyl}azepan-1-ium chloride

LC-MS: m/z=306.2 [M+H]⁺. ¹H NMR (400 MHz, DMSOd₆) δ 9.42 (bs, 1H), 7.41-7.32 (d, 2H), 7.23-7.14 (d, 2H), 5.96 (d, J=5.2 Hz, 1H), 4.27-4.18 (m, 1H), 3.32 (m, 2H), 3.10-2.95 (m, 3H), 2.50 (p, J=1.9 Hz, 1H), 2.35-2.27 (m, 2H), 2.19 (dq, J=31.4, 9.7, 9.2 Hz, 2H), 2.11-1.95 (m, 1H), 1.75 (dt, J=10.6, 5.6 Hz, 5H), 1.72-1.43 (m, 4H).

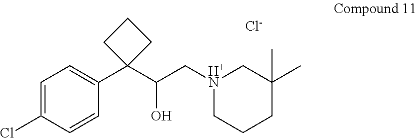

### 1-{2-[1-(3-Chlorophenyl)cyclobutyl]-2-hydroxyethyl}-3,3-dimethylpiperidin-1-ium chloride

LC-MS: m/z=322.2 [M+H]⁺. ¹H NMR (300 MHz, DMSOd₆) δ 8.43 (s, 1H), 7.43-7.28 (m, 2H), 7.18 (dq, J=8.6, 2.4, 2.0 Hz, 2H), 6.08 (d, J=5.2 Hz, 1H), 4.41-4.26 (m, 1H), 3.16 (d, J=10.9 Hz, 2H), 3.02-3.4 (m, 3H), 2.90-2.75 (m, 1H), 2.65 (dt, J=23.0, 11.6 Hz, 2H), 2.41 (d, J=14.7 Hz, 2H), 2.24 (dd, J=18.7, 6.8 Hz, 4H), 2.01 (dt, J=17.2, 8.8 Hz, 2H), 1.92-1.68 (m, 3H), 1.60 (d, J=14.1 Hz, 1H), 1.41 (d, J=13.2 Hz, 1H), 1.26 (d, J=12.5 Hz, 2H), 1.07 (d, J=11.4 Hz, 3H), 0.90 (d, J=13.8 Hz, 3H).

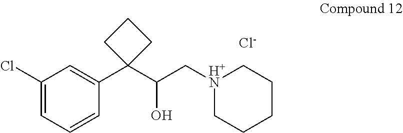

### 1-{2-[1-(3-Chlorophenyl)cyclobutyl]-2-hydroxyethyl}piperidin-1-ium chloride

LC-MS: m/z=294.2 [M+H]⁺. ¹H NMR (400 MHz, DMSOd₆) δ 9.34 (bs, 1H), 7.35 (t, J=7.8 Hz, 1H), 7.28 (dt, J=8.1, 1.5 Hz, 1H), 7.19 (t, J=1.9 Hz, 1H), 7.12 (dt, J=7.5, 1.4 Hz, 1H), 5.96 (d, J=5.1 Hz, 1H), 4.36-4.23 (m, 1H), 3.46 (d, J=12.0 Hz, 1H), 3.32 (d, 1H), 2.97 (dd, J=12.9, 6.4 Hz, 1H), 2.81 (dqd, J=12.5, 8.9, 5.3 Hz, 2H), 2.6 (m, 1H), 2.39-2.22 (m, 3H), 2.20-2.11 (m, 1H), 2.08-1.92 (m, 1H), 1.82-1.56 (m, 6H), 1.29 (q, J=17.4, 15.6 Hz, 1H).

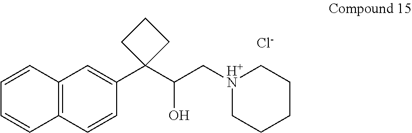

### 1-{2-hydroxy-2-[1-(naphthalen-2-yl)cyclobutyl]ethyl}piperidin-1-ium chloride

LC-MS: m/z=310.2 [M+H]⁺. ¹H NMR (400 MHz, DMSO d₆) δ 9.03 (bs, 1H), 7.94-7.79 (m, 3H), 7.68 (d, J=2.0 Hz, 1H), 7.54-7.44 (m, 2H), 7.36 (dd, J=8.5, 1.9 Hz, 1H), 6.00 (bs, 1H), 4.28 (s, 1H), 3.54-3.39 (m, 1H), 2.94-2.71 (m, 3H), 2.65-2.54 (m, 2H), 2.43-2.25 (m, 3H), 2.13-1.92 (m, 2H), 1.89-1.50 (m, 6H), 1.33-1.17 (m, 1H).

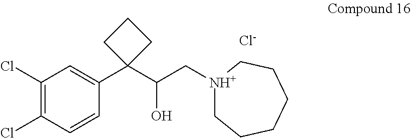 [SMILES: OC(C[NH+]1CCCCCC1)C1(c2ccc(Cl)c(Cl)c2)CCC1 ; [Cl-]]

### 1-{2-[1-(3,4-Dichlorophenyl)cyclobutyl]-2-hydroxyethyl}azepan-1-ium chloride

LC-MS: m/z=342.2 [M+H]⁺. ¹H NMR (300 MHz, DMSOd₆) δ 9.36 (bs, 1H), 7.57 (d, J=8.3 Hz, 1H), 7.37 (d, J=2.0 Hz, 1H), 7.15 (dd, J=8.4, 2.1 Hz, 1H), 6.00 (d, J=5.2 Hz, 1H), 4.26 (d, J=9.2 Hz, 1H), 3.48-3.38 (m, 1H), 3.33-3.24 (m, 1H), 3.18-2.95 (m, 3H), 2.43-2.22 (m, 3H), 2.22-2.08 (m, 1H), 2.06-1.91 (m, 1H), 1.76 (h, J=4.5 Hz, 5H), 1.66-1.38 (m, 5H).

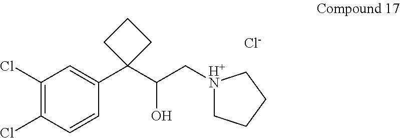

### 1-{2-[1-(3,4-Dichlorophenyl)cyclobutyl]-2-hydroxyethyl}pyrrolidin-1-ium chloride

LC-MS: m/z=314.1 [M+H]⁺. ¹H NMR (300 MHz, DMSOd₆) δ 9.57 (bs, 1H), 7.58 (d, J=8.3 Hz, 1H), 7.35 (d, J=2.0 Hz, 1H), 7.13 (dd, J=8.3, 2.1 Hz, 1H), 6.05 (bs, 1H), 4.15 (d, J=10.3 Hz, 1H), 3.39 (m, 2H), 3.18-3.01 (m, 1H), 2.95 (m, 3H), 2.29 (d, J=8.0 Hz, 3H), 2.19-1.98 (m, 2H), 1.98-1.67 (m, 7H).

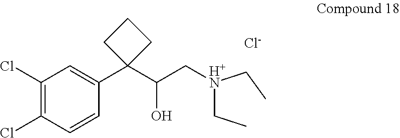

### {2-[1-(3,4-Dichlorophenyl)cyclobutyl]-2-hydroxyethyl}diethylazanium chloride

LC-MS: m/z=316.2 [M+H]⁺. ¹H NMR (300 MHz, DMSOd₆) δ 9.14 (bs, 1H), 7.59 (d, J=8.3 Hz, 1H), 7.41 (d, J=2.1 Hz, 1H), 7.18 (dd, J=8.4, 2.1 Hz, 1H), 6.02 (d, J=5.1 Hz, 1H), 4.21 (dd, J=9.3, 5.1 Hz, 1H), 3.25-2.99 (m, 4H), 2.95 (dd, J=13.1, 6.9 Hz, 1H), 2.47-2.09 (m, 5H), 1.99 (t, J=4.8 Hz, 1H), 1.77 (m, 1H), 1.10 (dt, J=12.2, 7.2 Hz, 6H).

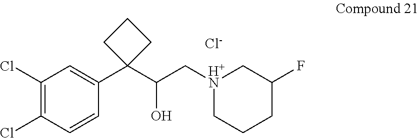

### 1-{2-[1-(3,4-Dichlorophenyl)cyclobutyl]-2-hydroxyethyl}-3-fluoropiperidin-1-ium chloride

LC-MS: m/z=346.1 [M+H]⁺. ¹H NMR (300 MHz, DMSOd₆) δ 10.00-9.25 (m, 1H), 7.58 (dd, J=8.3, 3.6 Hz, 1H), 7.36 (dd, J=3.8, 2.1 Hz, 1H), 7.14 (dt, J=8.1, 1.9 Hz, 1H), 6.05 (dd, J=12.3, 5.1 Hz, 1H), 5.85 (d, J=5.1 Hz, 1H), 5.15 (s, 0H), 4.97 (d, J=15.3 Hz, 1H), 4.79 (s, 0H), 4.40-4.11 (m, 1H), 3.86 (t, J=12.1 Hz, 1H), 3.58 (d, J=47.1 Hz, 1H), 3.42 (s, 1H), 3.23-2.74 (m, 4H), 2.45-2.21 (m, 4H), 2.17-1.80 (m, 5H), 1.74 (d, J=16.5 Hz, 4H), 1.20 (dd, J=15.1, 7.8 Hz, 1H).

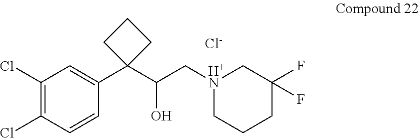

### 1-{2-[1-(3,4-Dichlorophenyl)cyclobutyl]-2-hydroxyethyl}-3,3-difluoropiperidin-1-ium chloride

LC-MS: m/z=364.1 [M+H]⁺. ¹H NMR (300 MHz, cd₃od) δ 7.49 (dd, J=8.2, 1.6 Hz, 1H), 7.40 (d, J=2.1 Hz, 1H), 7.16 (dd, J=8.3, 2.1 Hz, 1H), 4.30 (d, J=10.4 Hz, 1H), 3.84 (s, 1H), 3.48 (d, J=33.4 Hz, 1H), 3.05 (s, 2H), 2.71 (d, J=12.6 Hz, 1H), 2.57 (td, J=10.4, 5.6 Hz, 1H), 2.35 (dt, J=18.3, 8.8 Hz, 4H), 2.18-1.72 (m, 6H).

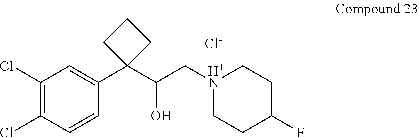

### 1-{2-[1-(3,4-Dichlorophenyl)cyclobutyl]-2-hydroxyethyl}-4-fluoropiperidin-1-ium chloride

LC-MS: m/z=346.1 [M+H]⁺. ¹H NMR (300 MHz, DMSOd₆) δ 9.50 (bs, 1H), 7.58 (d, J=8.3 Hz, 1H), 7.36 (dd, J=3.5, 2.0 Hz, 1H), 7.14 (dt, J=8.4, 2.4 Hz, 1H), 6.03 (t, J=3.9 Hz, 1H), 5.03-4.87 (m, 1H), 4.30 (dd, J=10.1, 5.0 Hz, 1H), 3.63-3.37 (m, 2H), 3.20-2.82 (m, 3H), 2.49-2.35 (m, 1H), 2.33-1.56 (m, 9H), 1.24 (s, 1H).

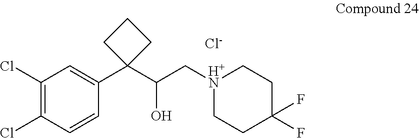

### 1-{2-[1-(3,4-Dichlorophenyl)cyclobutyl]-2-hydroxyethyl}-4,4-difluoropiperidin-1-ium chloride

LC-MS: m/z=364.3 [M+H]⁺. ¹H NMR (300 MHz, cd₃od) δ 7.48 (d, J=8.3 Hz, 1H), 7.39 (t, J=2.0 Hz, 1H), 7.15 (dt, J=7.7, 1.8 Hz, 1H), 4.30 (dd, J=11.0, 2.7 Hz, 1H), 3.64 (d, J=43.6 Hz, 2H), 3.30 (d, J=1.8 Hz, 1H), 3.10 (dd, J=13.1, 2.7 Hz, 1H), 2.72-2.47 (m, 2H), 2.46-2.18 (m, 7H), 2.18-1.96 (m, 1H), 1.89 (dtt, J=17.3, 7.2, 4.4 Hz, 1H), 1.29 (s, 1H).

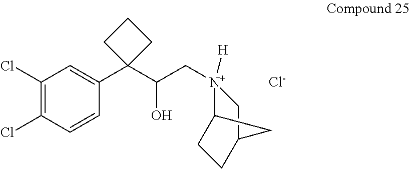 [SMILES: OC(C[NH+]1CC2CCC1C2)C1(c2ccc(Cl)c(Cl)c2)CCC1 ; [Cl-]]

### 2-{2-[1-(3,4-Dichlorophenyl)cyclobutyl]-2-hydroxyethyl}-2-azabicyclo[2.2.1]heptan-2-ium chloride

LC-MS: m/z=339.9 [M+H]⁺. ¹H NMR (300 MHz, DMSOd₆) δ 9.69-8.86 (m, 1H), 7.56 (dd, J=8.2, 2.3 Hz, 1H), 7.36 (dt, J=9.6, 1.8 Hz, 1H), 7.15 (ddt, J=11.0, 8.7, 2.3 Hz, 1H), 6.06-5.86 (m, 1H), 4.36-3.93 (m, 2H), 3.32 (m, 2H), 2.95 (m, 1H), 2.50 (m, 1H), 2.33-2.17 (m, 3H), 2.01 (m, 4H), 1.8-1.35 (m, 6H).

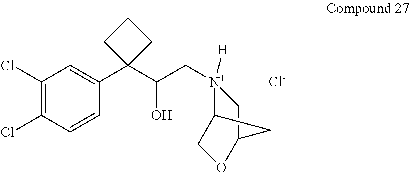 [SMILES: OC(C[NH+]1CC2CC1CO2)C1(c2ccc(Cl)c(Cl)c2)CCC1 ; [Cl-]]

### 5-{2-[1-(3,4-Dichlorophenyl)cyclobutyl]-2-hydroxyethyl}-2-oxa-5-azabicyclo[2.2.1]heptan-5-ium chloride

LC-MS: m/z=342.35 [M+H]⁺. ¹H NMR (300 MHz, DMSOd₆) δ 10.12-9.94 (m, 1H), 7.57 (dd, J=8.4, 3.4 Hz, 1H), 7.38 (dt, J=9.6, 1.8 Hz, 1H), 7.13 (d, J=7.3 Hz, 1H), 6.00 (dd, J=11.8, 5.1 Hz, 1H), 4.65-4.35 (m, 2H), 4.37-3.90 (m, 2H), 3.77-2.88 (m, 6H), 2.4-2.2 (m, 3H), 2.2-1.98 (m, 3H), 1.82 (m, 1H).

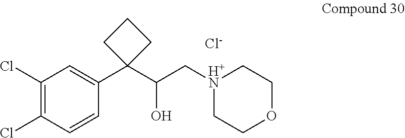

### 4-{2-[1-(3,4-Dichlorophenyl)cyclobutyl]-2-hydroxyethyl}morpholin-4-ium chloride

LC-MS: m/z=330.1 [M+H]⁺. ¹H NMR (300 MHz, DMSOd₆) δ 10.03 (m, 1H), 7.58 (dd, J=8.3 Hz, 1H), 7.36 (dt, J=9.6 Hz, 1H), 7.14 (dd, J=8.3, 2.2 Hz, 1H), 6.01 (d, J=5.1 Hz, 1H), 4.33 (m, 1H), 3.98-3.82 (m, 2H), 3.82-3.61 (m, 2H), 3.37 (m, 2H), 3.0 (m, 3H), 2.50 (p, J=1.8 Hz, 2H), 2.40 (q, J=8.4 Hz, 2H), 2.14 (q, J=9.56 Hz, 1H), 1.99 (q, J=9.3, 8.9, 1H), 1.78 (m, 1H).

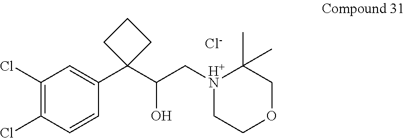

### 4-{2-[1-(3,4-dichlorophenyl)cyclobutyl]-2-hydroxyethyl}-3,3-dimethylmorpholin-4-ium chloride

LC-MS: m/z=357.9 [M+H]⁺. ¹H NMR (300 MHz, DMSOd₆) δ 10.42-9.38 (m, 1H), 7.57 (dd, J=8.3 Hz, 1H), 7.46 (dt, J=7.1, 2.1 Hz, 1H), 7.23 (dd, J=10.2, 5 Hz, 1H), 6.01-5.8 (m, 1H), 4.6-4.2 (m, 1H), 3.98-3.77 (m, 2H), 3.7 (m, 1H), 3.6-3.6 (m, 2H), 3.2 (m, 2H), 2.9 (m, 1H), 2.4-2.0 (m, 5H), 1.78 (m, 1H), 1.30-1.09 (m, 6H).

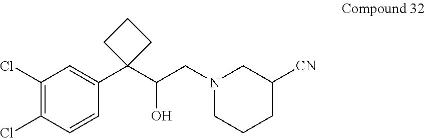 [SMILES: N#CC1CCCN(CC(O)C2(c3ccc(Cl)c(Cl)c3)CCC2)C1]

### 1-{2-[1-(3,4-dichlorophenyl)cyclobutyl]-2-hydroxyethyl}piperidine-3-carbonitrile

LC-MS: m/z=352.9 [M+H]⁺. ¹H NMR (400 MHz, CDCl₃) δ 7.41 (d, J=8.3 Hz, 1H), 7.36 (d, J=2.2 Hz, 1H), 7.06 (dd, J=8.4, 2.2 Hz, 1H), 3.9-3.8 (m, 1H), 3.6-3.3 (m, 2H), 2.80-2.75 (m, 2H), 2.6 (m, 2H), 2.28 (m, 3H), 2.24 (m, 2H), 2.12 (m, 2H), 2.00-1.8 (m, 5H).

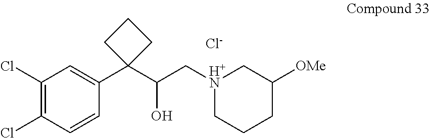

### 1-{2-[1-(3,4-Dichlorophenyl)cyclobutyl]-2-hydroxyethyl}-3-methoxypiperidin-1-ium chloride

LC-MS: m/z=358.1 (M+H]⁺. ¹H NMR (300 MHz, DMSOd₆) δ 9.6-8.9 (m, 1H), 7.58 (dd, J=8.3, 6.2 Hz, 1H), 7.39-7.31 (m, 1H), 7.17-7.15 (dd, J=8.4, 2.1 Hz, 1H), 6.02-5.92 (m, 1H), 4.28 (m, 1H), 3.88-3.2 (m, 6H), 3.0 (m, 3H), 2.8 (m, 1H), 2.4-2.0 (m, 5H), 1.90-1.70 (m, 4H), 1.4 (m, 1H).

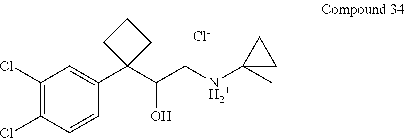 [SMILES: CC1([NH2+]CC(O)C2(c3ccc(Cl)c(Cl)c3)CCC2)CC1 ; [Cl-]]

### N-{2-[1-(3,4-Dichlorophenyl)cyclobutyl]-2-hydroxyethyl}-1-methylcyclopropan-1-aminium chloride

LC-MS: m/z=313.9 [M+H]⁺. ¹H NMR (300 MHz, DMSOd₆) δ 8.67 (m, 2H), 7.58 (d, J=8.3, 1H), 7.41 (d, J=2.1 Hz, 1H), 7.19 (dd, J=8.4, 2.1 Hz, 1H), 5.88 (d, J=4.9 Hz, 1H), 4.12 (m, 1H), 3.4 (m, 1H), 2.98 (m, 1H), 2.4-2.12 (m, 4H), 1.98 (m, 1H), 1.8 (m, 1H), 1.26 (s, 3H), 1.11-1.01 (m, 1H), 1.8 (m, 1H), 1.6 (m, 2H).

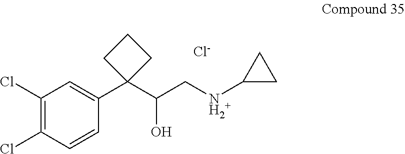 [SMILES: OC(C[NH2+]C1CC1)C1(c2ccc(Cl)c(Cl)c2)CCC1 ; [Cl-]]

### N-{2-[1-(3,4-Dichlorophenyl)cyclobutyl]-2-hydroxyethyl}cyclopropanaminium chloride

LC-MS: m/z=300.1 [M+H]⁺. ¹H NMR (300 MHz, DMSOd₆) δ 8.67 (m, 1H), 7.59 (d, J=8.3, 1H), 7.37 (d, J=2.1 Hz, 1H), 7.15 (dd, J=8.4, 2.1 Hz, 1H), 5.92 (d, J=4.8 Hz, 1H), 4.08 (m, 1H), 3.32 (m, 1H), 2.98 (m, 1H), 2.6 (m, 2H), 2.5-2.12 (p, J=1.9 Hz, 4H), 1.98 (m, 1H), 1.8 (m, 1H), 0.96 (m, 1H), 0.67 (m, 2H).

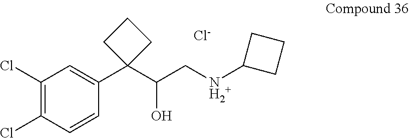 [SMILES: OC(C[NH2+]C1CCC1)C1(c2ccc(Cl)c(Cl)c2)CCC1 ; [Cl-]]

### N-{2-[1-(3,4-Dichlorophenyl)cyclobutyl]-2-hydroxyethyl}cyclobutanaminium chloride

LC-MS: m/z=313.9 [M+H]⁺. ¹H NMR (300 MHz, DMSOd₆) δ 8.62 (m, 2H), 7.58 (d, J=8.3, 1H), 7.41 (dd, J=8.3, 2.1 Hz, 1H), 7.36 (d, J=2.1 Hz, 1H), 5.93 (d, J=4.9 Hz, 1H), 4.05 (dd, J=9.6, 4.7 Hz, 1H), 3.61 (m, 1H), 2.73 (m, 1H), 2.28 (d, J=7.7 Hz, 3H), 2.21-1.83 (m, 7H), 1.81-1.59 (m, 3H).

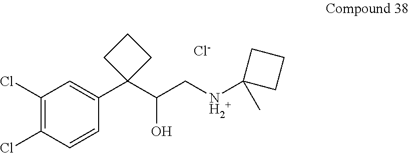 [SMILES: CC1([NH2+]CC(O)C2(c3ccc(Cl)c(Cl)c3)CCC2)CCC1 ; [Cl-]]

### N-{2-[1-(3,4-Dichlorophenyl)cyclobutyl]-2-hydroxyethyl}-1-methylcyclobutan-1-aminium chloride

LC-MS: m/z=327.9 [M+H]⁺. ¹H NMR (300 MHz, DMSOd₆) δ 8.90-8.75 (m, 1H), 8.6-8.45 (m, 1H), 7.57 (d, J=8.3, 1H), 7.41 (d, J=2.1 Hz, 1H), 7.17 (d, J=2.1 Hz, 1H), 5.91 (d, J=4.8 Hz, 1H), 4.09 (dd, J=9.6, 4.8 Hz, 1H), 2.86-2.67 (m, 6H), 2.09 (m, 3H), 1.88-1.67 (m, 5H), 1.31 (s, 3H).

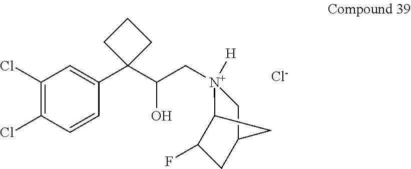 [SMILES: OC(C[NH+]1CC2CC(F)C1C2)C1(c2ccc(Cl)c(Cl)c2)CCC1 ; [Cl-]]

### 2-{2-[1-(3,4-Dichlorophenyl)cyclobutyl]-2-hydroxyethyl}-6-fluoro-2-azabicyclo[2.2.1]heptan-2-ium chloride

LC-MS: 358.1 [(M+H)-HCl]⁺. ¹H NMR (300 MHz, DMSOd₆) δ 10.4-9.6 (m, 1H), 7.56 (d, J=8.1 Hz, 1H), 7.41 (d, J=2.8 Hz, 1H), 7.22-7.08 (m, 1H), 6.02 (dd, J=13.6, 5.1 Hz, 1H), 5.56-5.1 (m, 1H), 4.54-4.20 (m, 2H), 3.23-3.03 (m, 2H), 2.96 (m, 1H), 2.67 (m, 2H), 2.4-2.2 (m, 2H), 2.2-2.0 (m, 2H), 1.98 (m, 3H), 1.8-1.6 (m, 3H).

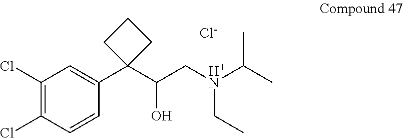

### {2-[1-(3,4-Dichlorophenyl)cyclobutyl]-2-hydroxyethyl}(ethyl)(propan-2-yl)azanium chloride

LC-MS: m/z=330.0 [M+H]⁺. ¹H NMR (300 MHz, DMSOd₆) δ 9.3-8.95 (m, 1H), 7.58 (d, J=8.4 Hz, 1H), 7.44 (d, J=2.3 Hz, 1H), 7.22 (dt, J=8.3, 2.4 Hz, 1H), 6.04 (d, J=5.1 Hz, 1H), 4.24 (m, 1H), 3.6 (m, 1H), 3.2-2.96 (m, 3H), 2.86 (m, 1H), 2.4-2.2 (m, 4H), 1.98 (m, 1H), 1.8-1.6 (m, 1H) 1.2-1 (m, 9H).

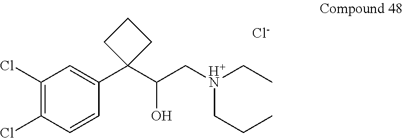

### {2-[1-(3,4-Dichlorophenyl)cyclobutyl]-2-hydroxyethyl}(ethyl)propylazanium chloride

LC-MS: 329.9 [M+H]⁺. ¹H NMR (400 MHz, DMSOd₆) δ 9.23 (s, 1H), 7.58 (d, J=8.3 Hz, 1H), 7.41 (d, J=2.0 Hz, 1H), 7.18 (dd, J=8.3, 2.1 Hz, 1H), 6.01 (d, J=5.2 Hz, 1H), 4.21 (d, J=12.1 Hz, 1H), 3.10 (m, 3H), 3.01-2.80 (m, 3H), 2.32 (ddd, J=32.5, 16.5, 6.1 Hz, 3H), 2.17 (dt, J=19.4, 9.7 Hz, 1H), 1.99 (q, J=9.0 Hz, 1H), 1.76 (dt, J=9.9, 5.1 Hz, 1H), 1.52 (dd, J=17.2, 9.5 Hz, 2H), 1.11 (dt, J=16.8, 7.1 Hz, 3H), 0.86 (dt, J=23.9, 7.3 Hz, 3H).

Compounds described in Examples 5-7 below were synthesized using the same general synthetic protocols described in Example 3, Compound 5, for Steps 1, 2 and 4. A modified synthetic protocol for Step 3 was used. The modified synthetic protocols are described below.

### Example 5

### Synthesis of tert-butyl({2-[1-(3,4-dichlorophenyl)cyclobutyl]-2-hydroxyethyl})azanium chloride (Compound 20)

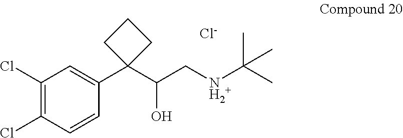 [SMILES: CC(C)(C)[NH2+]CC(O)C1(c2ccc(Cl)c(Cl)c2)CCC1 ; [Cl-]]

LC-MS: m/z=316.1 [M+H]⁺ [free base]. ¹H NMR (400 MHz, DMSOd₆) δ 8.52 (bs, 1H), 8.19 (s, 1H), 7.57 (d, J=8.3 Hz, 1H), 7.43 (d, J=2.1 Hz, 1H), 7.21 (dd, J=8.3, 2.1 Hz, 1H), 5.90 (d, J=4.6 Hz, 1H), 4.11 (dd, J=9.3, 4.5 Hz, 1H), 2.89 (t, J=10.8 Hz, 1H), 2.55-2.51 (m, 1H), 2.30 (t, J=7.9 Hz, 2H), 2.18 (ddd, J=11.6, 9.6, 7.7 Hz, 1H), 1.99-1.84 (m, 2H), 1.78 (dt, J=12.3, 5.6 Hz, 1H), 1.20 (s, 9H).

Modified Step 3:

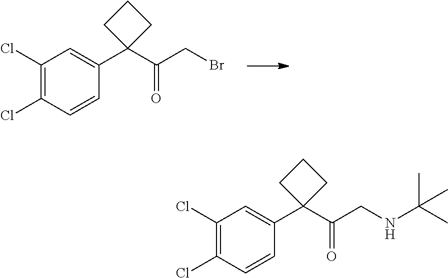 [SMILES: O=C(CBr)C1(c2ccc(Cl)c(Cl)c2)CCC1 ; CC(C)(C)NCC(=O)C1(c2ccc(Cl)c(Cl)c2)CCC1]

A stirred mixture of 2-bromo-1-[1-(3,4-dichlorophenyl)cyclobutyl]ethan-1-one (500 mg, 1.55 mmol) and t-butyl amine (10 mL) was heated to reflux in a sealed tube for 4 h. The reaction mixture was cooled to ambient temperature and quenched with ice cold water (10 mL), then extracted with EtOAc (2×15 mL). The combined EtOAc layers were dried over Na₂SO₄ and concentrated to dryness to obtain 2-(tert-butylamino)-1-[1-(3,4-dichlorophenyl)cyclobutyl]ethan-1-one (400 mg crude; LC-MS: m/z=314.3 [M+H]⁺), which was used in Step 4 without further purification.

### Example 6

### Synthesis of {2-[1-(3,4-dichlorophenyl)cyclobutyl]-2-hydroxyethyl}(propan-2-yl)azanium chloride (Compound 19)

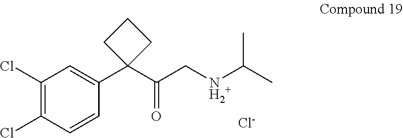 [SMILES: CC(C)[NH2+]CC(=O)C1(c2ccc(Cl)c(Cl)c2)CCC1 ; [Cl-]]

LC-MS: m/z=302.1 [M+H]⁺[free base]. ¹H NMR (300 MHz, DMSOd₆) δ 8.42 (bs, 1H), 8.19 (bs, 1H), 7.59 (d, J=8.3 Hz, 1H), 7.41 (d, J=2.1 Hz, 1H), 7.18 (dd, J=8.4, 2.1 Hz, 1H), 6.02 (d, J=5.1 Hz, 1H), 4.21 (dd, J=9.3, 5.1 Hz, 1H), 3.25 (m, 1H), 2.95 (dd, J=13.1, 6.9 Hz, 1H), 2.47-2.09 (m, 5H), 1.99 (t, J=4.8 Hz, 1H), 1.77 (m, 1H), 1.20 (d, j=12.2, 7.2 Hz, 3H) 1.10 (d, J=12.2, 7.2 Hz, 3H).

### Example 7

### Synthesis of 2-{2-[1-(3-Chlorophenyl)cyclobutyl]-2-hydroxyethyl}-2-azabicyclo[2.2.1]heptan-2-ium chloride (Compound 9)

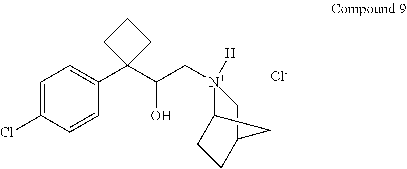 [SMILES: OC(C[NH+]1CC2CCC1C2)C1(c2ccc(Cl)cc2)CCC1 ; [Cl-]]

LC-MS: m/z=306.2 [M+H]⁺ [free base]. ¹H NMR (300 MHz, DMSOd₆) δ 9.43-8.6 (m, 1H), 7.44-7.24 (m, 2H), 7.23-7.06 (m, 2H), 5.99-5.82 (m, 1H), 4.08-3.99 (m, 2H), 3.22 (dd, J=11.1, 6.2 Hz, 1H), 3.00 (d, J=37.5 Hz, 3H), 2.27 (m, 4H), 1.65-1.38 (m, 8H), 1.29 (d, J=31.3 Hz, 1H).

Modified Step 3:

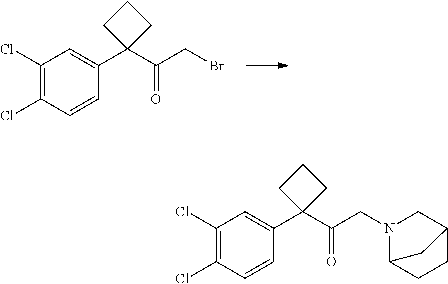 [SMILES: O=C(CBr)C1(c2ccc(Cl)c(Cl)c2)CCC1 ; O=C(CN1CC2CCC1C2)C1(c2ccc(Cl)c(Cl)c2)CCC1]

To a stirred solution of compound 2-bromo-1-[1-(3,4-dichlorophenyl)cyclobutyl]ethan-1-one (500 mg, 1.55 mmol) and 2-azabicyclo[2.2.1]heptane.HCl (250 mg, 1.86 mmol) in CH₂Cl₂ (5 mL) was added Et₃N (1 mL, 7.76 mmol) at 0° C. and the reaction stirred at RT for 16 h. The reaction mixture was quenched with ice cold water (10 mL) and extracted with EtOAc (2×15 mL). The combined EtOAc layer was dried over Na₂SO₄ and concentrated to dryness to give 2-{2-azabicyclo[2.2.1]heptan-2-yl}-1-[1-(3,4-dichlorophenyl)cyclobutyl]ethan-1-one (300 mg crude; LC-MS: m/z=304.1 [M+H]⁺) which was used in Step 4 without further purification.

### Example 8

### Synthesis of {2-[1-(3,4-dichlorophenyl)cyclobutyl]-2-hydroxyethyl}(1-fluoropropan-2-yl)azanium chloride (Compound 29)

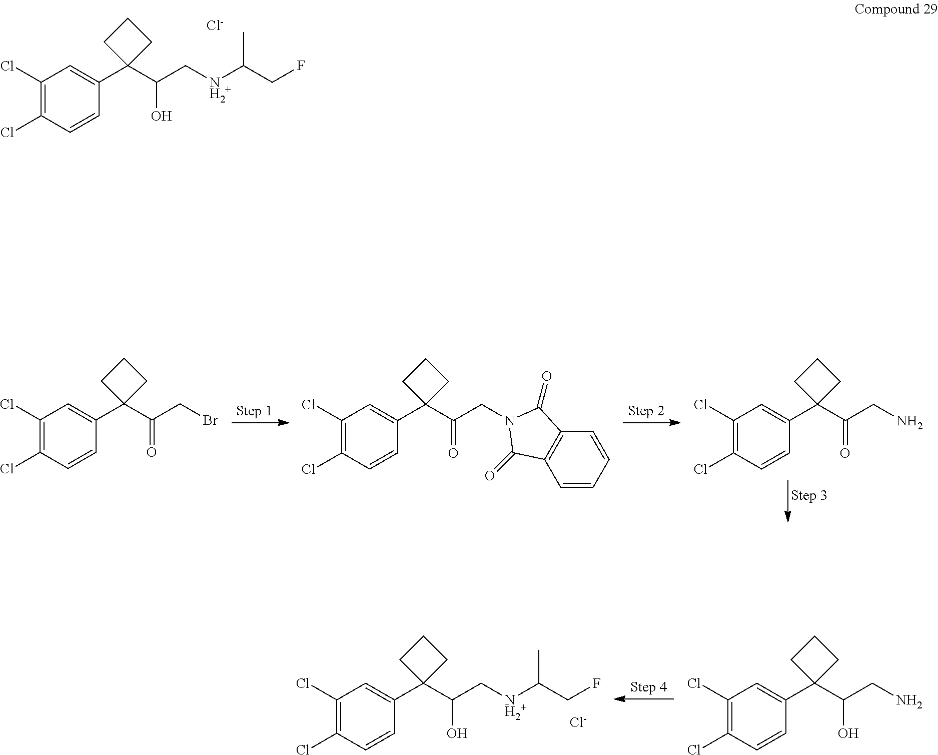

### Step 1: Synthesis of 2-{2-[1-(3,4-dichlorophenyl)cyclobutyl]-2-oxoethyl}-2,3-dihydro-1H-isoindole-1,3-dione

To a stirred solution of 2-bromo-1-(1-(3,4-dichlorophenyl)cyclobutyl)ethan-1-one (2.0 g, 6.21 mmol) in DMF (20 mL) was added potassium phtalimide (5.75 g, 31.05 mmol) portion wise at 0° C. The reaction was stirred at 50° C. for 2 h then cooled to ambient temperature and quenched with ice cold water (20 mL) over a period of 10 minutes, while maintaining the reaction mixture temperature below 15° C. The precipitate was filtered, washed with ice cold water (25 mL) and dried under vacuum to yield the title compound (1.7 g, 70% yield) as an off white solid. ¹H NMR (400 MHz, CDCl₃) δ 7.84 (dt, J=7.6, 3.7 Hz, 2H), 7.73 (dd, J=5.4, 3.0 Hz, 2H), 7.50 (d, J=8.3 Hz, 1H), 7.42 (d, J=2.2 Hz, 1H), 7.12 (dd, J=8.3, 2.2 Hz, 1H), 4.2 (s, 2H), 2.92 (ddd, J=14.9, 7.1, 3.8 Hz, 2H), 2.46 (tdd, J=9.6, 7.6, 2.1 Hz, 2H), 2.04 (dt, J=11.3, 8.4 Hz, 1H), 1.99-1.85 (m, 1H).

### Step 2: Synthesis of 2-amino-1-[1-(3,4-dichlorophenyl)cyclobutyl]ethan-1-one

To a stirred solution of 2-{2-[1-(3,4-dichlorophenyl)cyclobutyl]-2-oxoethyl}-2,3-dihydro-1H-isoindole-1,3-dione (1.0 g, 2.55 mmol) in EtOH (10 mL) was added NH₂NH₂.H₂O (0.3 mL, 5.68 mmol) portion wise at 0° C. The reaction was refluxed at 70° C. for 3 h then cooled to ambient temperature and quenched with 10% NaHCO₃ solution over a period of 10 minutes, while maintaining the reaction mixture temperature below 15° C. EtOH was distilled from the reaction mixture and the aqueous layer was extracted with CH₂Cl₂ (2×25 mL), dried over Na₂SO₄ and concentrated to dryness to give the title compound (600 mg, crude) as light brown liquid, which was used rapidly in the next step without further purification, due to its instability.

### Step 3: Synthesis of 2-amino-1-[1-(3,4-dichlorophenyl)cyclobutyl]ethan-1-ol

To a stirred solution of 2-amino-1-[1-(3,4-dichlorophenyl)cyclobutyl]ethan-1-one (600 mg, 2.32 mmol) in EtOH (10 mL) was added NaBH₄ (170 mg, 4.65 mmol) portionwise at 0° C. The reaction was stirred for 2 h then quenched with ice cold water over a period of 10 minutes, while maintaining the reaction mixture temperature below 15° C. Ethanol was distilled from the reaction mixture and the aqueous layer extracted with CH₂Cl₂ (2×25 mL), the combined organic layers dried over Na₂SO₄ and concentrated to dryness, to give the title compound (120 mg, crude) as a colorless viscous liquid. LC-MS: m/z=260.2 [M+H]⁺.

### Step 4: Synthesis of {2-[1-(3,4-dichlorophenyl)cyclobutyl]-2-hydroxyethyl}(1-fluoropropan-2-yl)azanium chloride (Compound 29)

To a stirred solution of 2-amino-1-[1-(3,4-dichlorophenyl)cyclobutyl]ethan-1-ol (120 mg, 0.46 mmol) in CH₂Cl₂ (10 mL) was added 1-fluoropropan-2-one (50 mg, 0.55 mmol) and Na(OAc)₃BH (292 mg, 1.38 mmol) portionwise at 0° C. The reaction was stirred for 2 h then quenched with ice cold water over a period of 10 min, while maintaining the reaction mixture temperature below 15° C. Ethanol was distilled from the reaction mixture and the aqueous layer was extracted with CH₂Cl₂ (2×25 mL), the combined organic layers dried over Na₂SO₄ and concentrated to dryness to give 1-[1-(3,4-dichlorophenyl)cyclobutyl]-2-[(1-fluoropropan-2-yl)amino]ethan-1-ol (110 mg, crude) as a colorless viscous liquid. LC-MS: m/z=320.3 [M+H]⁺.

To a stirred solution of 1-[1-(3,4-dichlorophenyl)cyclobutyl]-2-[(1-fluoropropan-2-yl)amino]ethan-1-ol (110 mg, 0.34 mmol) in 1,4-dioxane (2 mL) was added 4M HCl in dioxane (2 mL) at 0° C. and the mixture stirred at room temperature for 16 h. The mixture was concentrated to dryness and washed with Et₂O (10 mL) and EtOAc (10 mL). The organic solvent was decanted and the resulting solid was dried under vacuum to afford the title compound (38.8 mg) as an off white solid. LC-MS: m/z=320.3 [M+H]⁺. ¹H NMR (300 MHz, DMSOd₆) δ 9.2-9.0 (m, 1H), 8.7-8.6 (m, 1H), 7.56 (dd, J=8.2, 2.3 Hz, 1H), 7.36 (dt, J=9.6, 1.8 Hz, 1H), 7.15 (ddt, J=11.0, 8.7, 2.3 Hz, 1H), 6.06-5.86 (m, 1H), 4.8-4.4 (m, 2H), 4.2 (m, 1H), 3.6 (m, 1H), 1.98 (m, 2H), 2.4-2.1 (m, 4H), 1.98 (m, 1H), 1.8 (m, 1H), 1.2 (m, 3H).

### Example 9

### Synthesis of {2-[1-(3,4-dichlorophenyl)cyclobutyl]-2-hydroxyethyl}(3-fluorobutan-2-yl)azanium chloride (Compound 42)

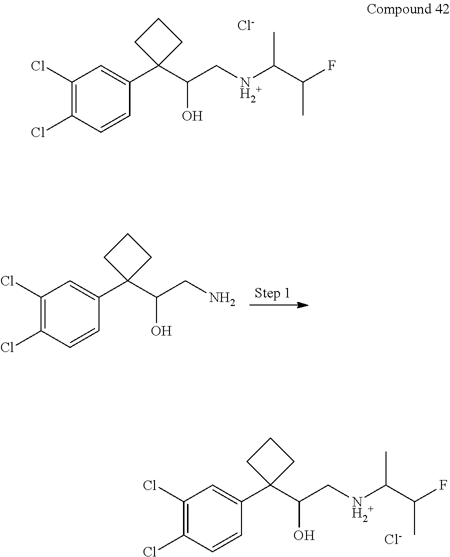 [SMILES: CC(F)C(C)[NH2+]CC(O)C1(c2ccc(Cl)c(Cl)c2)CCC1 ; [Cl-] ; NCC(O)C1(c2ccc(Cl)c(Cl)c2)CCC1 ; CC(F)C(C)[NH2+]CC(O)C1(c2ccc(Cl)c(Cl)c2)CCC1 ; [Cl-]]

### Step 1: Synthesis of {2-[1-(3,4-dichlorophenyl)cyclobutyl]-2-hydroxyethyl}(3-fluorobutan-2-yl)azanium chloride (Compound 42)

To a stirred solution of 2-amino-1-[1-(3,4-dichlorophenyl)cyclobutyl]ethan-1-ol (100 mg, 0.38 mmol) in CH₂Cl₂ (10 mL) was added 3-fluorobutan-2-one (0.04 g, 0.46 mmol) and Na(OAc)₃BH (240 mg, 1.14 mmol) portion wise at 0° C. The reaction was stirred for 2 h then quenched with ice cold water over a period of 10 min, while maintaining the reaction mixture temperature below 15° C. Ethanol was distilled from the reaction mixture and the aqueous layer was extracted with CH₂Cl₂ (2×25 mL), the combined organic layers dried over Na₂SO₄ and concentrated to dryness to give 1-[1-(3,4-dichlorophenyl)cyclobutyl]-2-[(3-fluorobutan-2-yl)amino]ethan-1-ol (90 mg, crude) as a colorless viscous liquid. To a stirred solution of 1-[1-(3,4-dichlorophenyl)cyclobutyl]-2-[(3-fluorobutan-2-yl)amino]ethan-1-ol (90 mg, 0.26 mmol) in 1,4-dioxane (2 mL) was added 4M HCl in dioxane (2 mL) at 0° C. and the mixture stirred at room temperature for 16 h. The mixture was concentrated to dryness and washed with Et₂O (10 mL) and EtOAc (10 mL). The organic solvent was decanted and the resulting solid was dried under vacuum to afford the title compound (7.8 mg) as an off white solid. LC-MS: m/z=334.3 [M+H]⁺. ¹H NMR (300 MHz, DMSOd₆) δ 8.8-8.4 (m, 2H), 7.58 (dd, J=8.3, 2.1 Hz, 1H), 7.38 (dt, J=4.1, 2.1 Hz, 1H), 7.24-7.12 (m, 1H), 5.92 (m, 1H), 5.2-4.6 (m, 1H), 4.14 (m, 1H), 3.4 (m, 2H), 3.0 (m, 2H), 2.4-2.0 (m, 4H), 1.98 (m, 1H), 1.4-1.2 (m, 3H), 1.2-1.0 (m, 3H).

### Example 10

### Synthesis of 1-{2-[1-(3,4-dichlorophenyl)cyclobutyl]-2-methoxyethyl}piperidin-1-ium chloride (Compound 59)

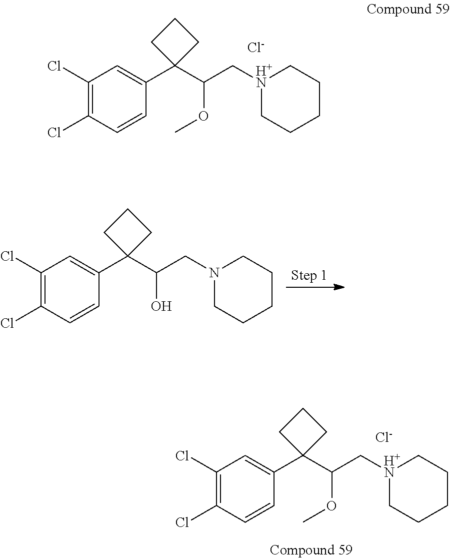

### Step 1: 1-{2-[1-(3,4-dichlorophenyl)cyclobutyl]-2-methoxyethyl}piperidin-1-ium chloride (Compound 59)

To a stirred solution of 1-(1-(3,4-dichlorophenyl)cyclobutyl)-2-(piperidin-1-yl)ethanol (Compound 7; 200 mg, 0.60 mmol, 1 eq) in THF was added methyl iodide (420 mg, 3.04 mmol, 5 eq) and potassium tert-butoxide (100 mg, 0.9 mmol, 1.5 eq) at 0° C. After stirring at room temperature for 3 h, the reaction mixture was carefully added to ice cold water (10 mL) over a period of 10 min, while maintaining the reaction mixture temperature below 15° C. The resulting mixture was extracted with EtOAc (2×10 mL). The combined EtOAc layers were washed with brine, the organic layer dried over Na₂SO₄ and concentrated under reduced pressure to give crude product as yellow oil. Purification by preparative HPLC gave 1-(2-(1-(3,4-dichlorophenyl)cyclobutyl)-2-methoxyethyl)piperidine (60 mg, 30% yield) as a pale yellow oil. LC-MS: m/z=342.1[M+H]⁺. A solution of 1-(2-(1-(3,4-dichlorophenyl)cyclobutyl)-2-methoxyethyl) piperidine (60 mg, 0.17 mmol, 1 eq) in 1,4-dioxane (0.2 mL) was treated with 4M HCl in dioxane (0.3 mL) at 0° C. After stirring at room temperature for 16 h, the mixture was concentrated to dryness and the residue washed with Et₂O (10 mL) then EtOAc (10 mL). The organic solvent was decanted off and the resulting solid dried under vacuum to afford the title compound (40 mg, 60% yield) as an off-white solid. LC-MS: m/z=342.1[M+H]⁺ (Free base). ¹H NMR (400 MHz, DMSOd₆) δ 9.41 (bs, 1H), 7.62 (d, J=8.3 Hz, 1H), 7.52 (d, J=2.2 Hz, 1H), 7.29 (dd, J=8.3, 2.2 Hz, 1H), 4.12 (d, J=9.7 Hz, 1H), 3.59 (s, 3H), 3.47-3.35 (m, 1H), 3.32-3.22 (m, 1H), 3.02-2.93 (m, 1H), 2.91-2.72 (m, 2H), 2.59 (d, J=10.6 Hz, 1H), 2.43-2.17 (m, 4H), 1.93 (dd, J=7.5, 3.6 Hz, 1H), 1.82-1.55 (m, 6H), 1.30 (dd, J=28.2, 15.6 Hz, 1H).

### Example 11

### Synthesis of 1-{2-[1-(3,4-dichlorophenyl)cyclopentyl]-2-oxoethyl}piperidin-1-ium chloride (Compound 61) and 1-{2-[1-(3,4-dichlorophenyl)cyclopentyl]-2-hydroxyethyl}piperidin-1-ium chloride (Compound 60)

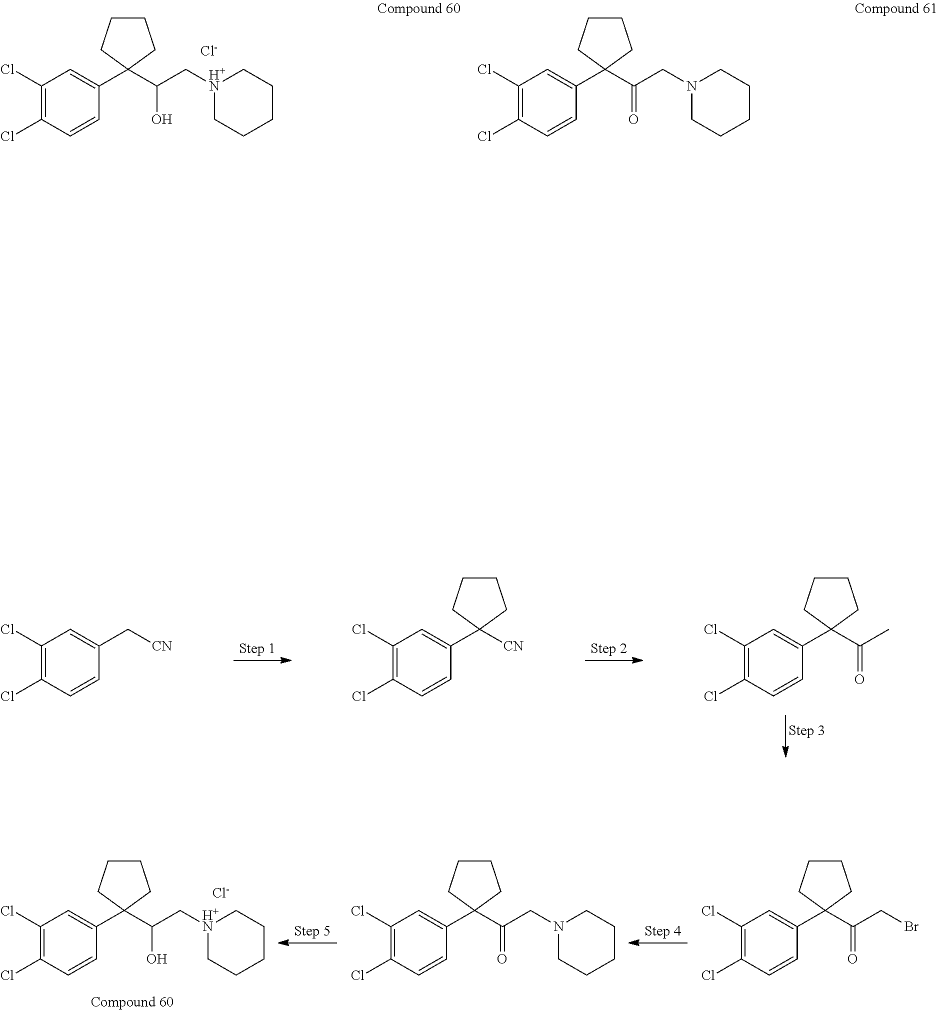

### Step 1: Synthesis of 1-(3,4-dichlorophenyl)cyclopentane-1-carbonitrile

To a stirred slurry of 60% NaH (4.7 g, 118.27 mmol) in DMSO (20 mL) was added a mixture of 2-(3,4-dichlorophenyl)acetonitrile (10 g, 53.76 mmol) and 1,3-dibromo butane (12.7 g, 59.13 mmol) in Et₂O (30 mL) dropwise. The reaction was stirred at room temperature for 18 h then cooled to 0° C. and quenched with 2-propanol (25 mL) and diluted with water (25 mL). The aqueous layer was extracted with hexane (2×100 mL) and the combined organic layers were dried over Na₂SO₄ and concentrated under reduced pressure to afford the crude product, which was purified by Silica chromatography (eluted with 5% ethyl acetate/hexane) to afford the title compound (10.0 g, 77.5% yield) as a pale yellow oil. ¹H NMR (400 MHz, CDCl₃) δ 7.53 (s, 1H), 7.45 (d, J=8.6 Hz, 1H), 7.35-7.11 (m, 1H), 2.48 (dhept, J=10.7, 4.0, 3.4 Hz, 2H), 2.17-1.80 (m, 6H).

### Step 2: Synthesis of 1-[1-(3,4-dichlorophenyl)cyclopentyl]ethan-1-one

To a stirred solution of 1-(3,4-dichlorophenyl)cyclopentane-1-carbonitrile (5.0 g, 20.8 mmol) in toluene (25 mL) was slowly added methyl magnesium bromide (3M in Et₂O; 20.7 mL, 62.4 mmol) at 10° C. The reaction was stirred at 75° C. for 16 h then cooled to 0° C. and poured onto crushed ice and quenched slowly with 6N HCl (20 mL), maintaining the temperature below 25° C. The resulting slurry was heated to 95° C. and stirred for 2 h, then cooled to room temperature and extracted with Et₂O (2×100 mL). The combined ether layers were washed with brine (25 mL), dried over Na₂SO₄ and concentrated under reduced pressure to afford the title compound (4.0 g, 75% yield) as an orange oil, which was used in the next step without further purification. ¹H NMR (400 MHz, CDCl₃) δ 7.44-7.30 (m, 2H), 7.10 (dd, J=8.4, 2.2 Hz, 1H), 2.47 (dddd, J=12.8, 5.5, 3.8, 1.9 Hz, 2H), 1.95 (s, 3H), 1.91-1.74 (m, 2H), 1.78-1.58 (m, 4H).

### Step 3: Synthesis of 2-bromo-1-[1-(3,4-dichlorophenyl)cyclopentyl]ethan-1-one

To a stirred solution of 1-(1-(3,4-dichlorophenyl)cyclopentyl)ethan-1-one (2.0 g, 7.78 mmol) in MeOH (25 mL) was added HBr (30% in AcOH; 0.08 mL, 0.31 mmol) and bromine (2.22 g, 14.0 mmol) at 0° C. After stifling at 0° C. for 16 h, the reaction mixture was carefully added to water (25 mL) over a period of 10 min, while maintaining the reaction mixture temperature below 15° C. The resulting mixture was extracted with Et₂O (2×25 mL). The combined ether layers were washed with brine (25 mL) and dried over Na₂SO₄ then concentrated under reduced pressure to give the title compound (2.0 g, crude) as an orange oil, which was used in the next step without further purification. ¹H NMR (400 MHz, CDCl₃) δ 7.47-7.33 (m, 2H), 7.12 (ddd, J=10.7, 8.4, 2.2 Hz, 1H), 3.87 (s, 2H), 2.63-2.37 (m, 2H), 2.01-1.84 (m, 2H), 1.84-1.62 (m, 4H).

### Step 4: Synthesis of 1-[1-(3,4-dichlorophenyl)cyclopentyl]-2-(piperidin-1-yl)ethan-1-one

To a stirred solution of piperidine (600 mg, 7.10 mmol) and potassium carbonate (4.08 g, 29.6 mmol) in acetone (10 mL) was added a slurry of 2-bromo-1-[1-(3,4-dichlorophenyl)cyclopentyl]ethan-1-one (2.0 g, 5.92 mmol) and sodium iodide (1.0 g, 7.10 mmol) in acetone (1 mL). The mixture was heated to reflux for 16 h, then cooled to ambient temperature, quenched with water (10 mL) and extracted with EtOAc (2×25 mL). The combined organic layer was dried over Na₂SO₄, concentrated to dryness and purified by silica gel chromatography (eluting with 10-12% EtOAc/hexane) to afford the title compound (1.0 g, 50% yield) as a pale yellow oil. LC-MS: m/z=340.1 [M+H]⁺.

### Step 5: Synthesis of 1-{2-[1-(3,4-dichlorophenyl)cyclopentyl]-2-hydroxyethyl}piperidin-1-ium chloride

To a stirred solution of 1-(1-(3,4-dichlorophenyl)cyclopentyl)-2-(piperidin-1-yl)ethan-1-one (800 mg, 2.35 mmol) in MeOH (20 mL) was added NaBH₄ (133 mg, 3.52 mmol) portion wise at 0° C. After stirring at 0° C. for 2 h the reaction mixture was quenched with ice cold water over a period of 10 min, while maintaining the reaction mixture temperature below 15° C. MeOH was then distilled from reaction mixture under reduced pressure. The resulting mixture was extracted with CH₂Cl₂ (2×15 mL). The combined CH₂Cl₂ layers were dried over Na₂SO₄, concentrated to dryness and purified by silica gel chromatography (10-15% EtOAc/hexane) to afford 1-(1-(3,4-dichlorophenyl)cyclopentyl)-2-(piperidin-1-yl)ethanol (600 mg, 75% yield) as an off white solid. LC-MS: m/z=342.2 [M+H]⁺.

To a stirred solution of 1-(1-(3,4-dichlorophenyl)cyclopentyl)-2-(piperidin-1-yl)ethanol (600 mg, 1.75 mmol) in 1,4-dioxane (2 mL) was added 4M HCl in dioxane (2 mL) at 0° C. After stirring at room temperature for 16 h the reaction mixture was concentrated to dryness and washed with Et₂O (10 mL) and EtOAc (10 mL). The organic solvent was decanted and the resulting solid was dried under vacuum to afford the title compound (510 mg, 77% yield) as an off white solid. LC-MS: m/z=342.2 [M+H]⁺. ¹H NMR (300 MHz, DMSOd₆) δ 9.09 (bs, 1H), 7.57 (dd, J=5.3, 3.2 Hz, 2H), 7.34 (dd, J=8.5, 2.2 Hz, 1H), 5.96 (d, J=5.3 Hz, 1H), 4.10-3.93 (m, 1H), 3.50-3.36 (m, 1H), 3.30-3.19 (m, 1H), 2.93 (dd, J=13.0, 7.1 Hz, 1H), 2.87-2.64 (m, 2H), 2.39 (ddd, J=13.7, 10.6, 3.6 Hz, 1H), 2.09 (dt, J=12.6, 7.1 Hz, 1H), 1.88 (dp, J=12.4, 4.9, 3.6 Hz, 3H), 1.81-1.56 (m, 7H), 1.48 (p, J=7.9 Hz, 2H), 1.38-1.26 (m, 1H).

### Example 12

### Synthesis of 1-{2-[1-(3,4-dichlorophenyl)cyclobutyl]-2-hydroxypropyl}piperidin-1-ium chloride (Compound 71)

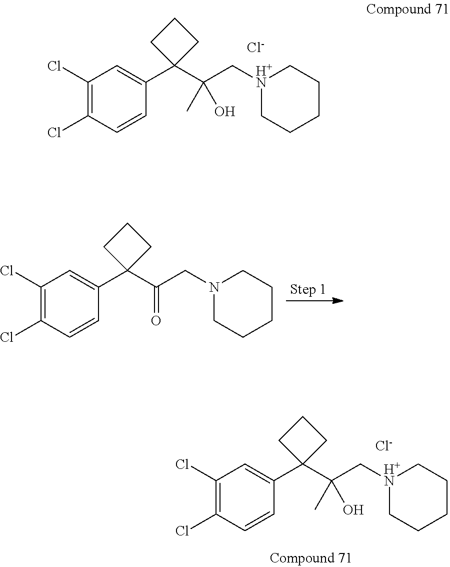

### Step 1: 1-{2-[1-(3,4-dichlorophenyl)cyclobutyl]-2-hydroxypropyl}piperidin-1-ium chloride (Compound 71)

To a stirred solution of 1-(1-(3,4-dichlorophenyl)cyclobutyl)-2-(piperidin-1-yl)ethanone (500 mg, 1.53 mmol) in THF was slowly added methyl magnesium bromide (3M in Et₂O) (2.5 mL, 7.65 mmol) at 10° C. After stirring at room temperature for 48 h the reaction mixture was cooled to 0° C., then poured onto crushed ice and extracted with EtOAc (2×25 mL). The combined EtOAc layers were washed with brine (10 mL), dried over Na₂SO₄ and concentrated under reduced pressure to give crude product. Purification by Mass-Directed preparative HPLC gave 2-(1-(3,4-dichlorophenyl)cyclobutyl)-1-(piperidin-1-yl)propan-2-ol (90 mg, 18% yield) as pale yellow oil. A solution of 2-(1-(3,4-dichlorophenyl)cyclobutyl)-1-(piperidin-1-yl)propan-2-ol in 1,4-dioxane (0.5 ml) was treated with 4M HCl in 1,4-dioxane (0.5 ml) at 0° C. After stirring at room temperature for 16 h, the mixture was concentrated to dryness and the residue washed with Et₂O (10 mL) then EtOAc (10 mL). The organic solvent was decanted off and the resulting solid dried under vacuum to afford the title compound (21 mg, 25%) as a pale brown solid. LC-MS: m/z=342.2 [M+H] (Free base). ¹H NMR (400 MHz, DMSOd₆) δ 8.63 (bs, 1H), 7.56 (d, J=8.4 Hz, 1H), 7.55 (d, J=2.1 Hz, 1H), 7.31 (dd, J=8.5, 2.2 Hz, 1H), 5.62 (s, 1H), 3.63-3.48 (m, 1H), 3.44-3.37 (m, 1H), 3.10 (dd, J=13.4, 4.9 Hz, 1H), 3.03-2.82 (m, 2H), 2.76-2.56 (m, 3H), 2.20 (ddd, J=12.3, 9.5, 5.5 Hz, 2H), 1.77 (qd, J=10.4, 10.0, 4.4 Hz, 4H), 1.70-1.53 (m, 3H), 1.44-1.28 (m, 1H), 1.20 (s, 3H).

### Example 13

### Syntheses of 1-[(2S)-2-[1-(3,4-dichlorophenyl)cyclobutyl]-2-hydroxyethyl]piperidin-1-ium chloride (Compound 13) and 1-[(2R)-2-[1-(3,4-dichlorophenyl)cyclobutyl]-2-hydroxyethyl]piperidin-1-ium chloride (Compound 14)

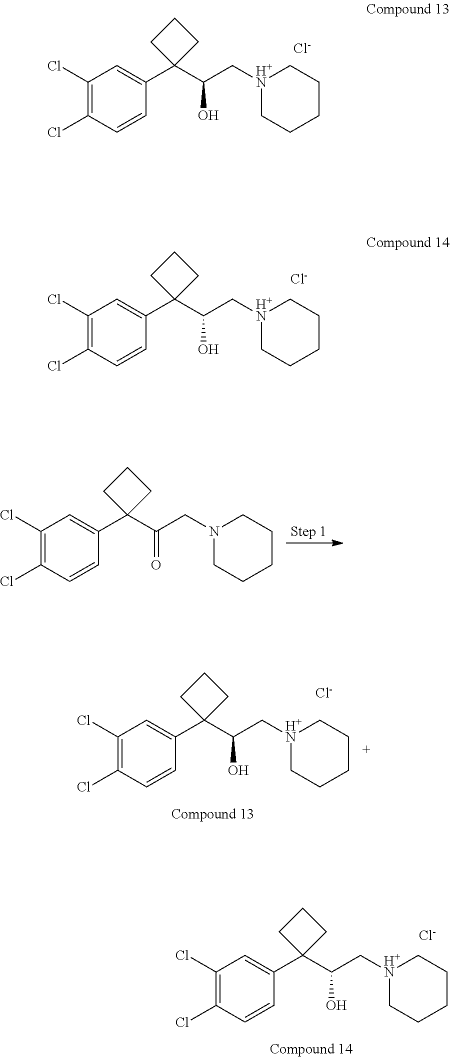

### Step 1: Synthesis of 1-[(2S)-2-[1-(3,4-dichlorophenyl)cyclobutyl]-2-hydroxyethyl]piperidin-1-ium chloride (Compound 13) and 1-[(2R)-2-[1-(3,4-dichlorophenyl)cyclobutyl]-2-hydroxyethyl]piperidin-1-ium chloride (Compound 14)

To a stirred solution of 1-(1-(3,4-dichlorophenyl)cyclobutyl)-2-(piperidin-1-yl)ethan-1-one (1.8 g, 5.52 mmol) in MeOH (25 mL) was added NaBH₄ (310 mg, 8.28 mmol) portion wise at 0° C. and the reaction stirred at 0° C. for 2 h. The reaction mixture was quenched with ice cold water over a period of 10 min, while maintaining the reaction mixture temperature below 15° C. MeOH was distilled from reaction mixture under reduced pressure. The resulting mixture was extracted with CH₂Cl₂ (2×25 mL). The combined CH₂Cl₂ layers were dried over Na₂SO₄, concentrated to dryness and purified by silica gel chromatography (10-15% EtOAc/hexane) to afford 1-(1-(3,4-dichlorophenyl)cyclobutyl)-2-(piperidin-1-yl)ethanol (1.8 g, 100%) as an off white solid. LC-MS: m/z=328.2 [M+H]⁺.

A 1.0 g sample of 1-(1-(3,4-dichlorophenyl)cyclobutyl)-2-(piperidin-1-yl)ethanol was separated into its individual enantiomers, (1R)-1-[1-(3,4-dichlorophenyl)cyclobutyl]-2-(piperidin-1-yl)ethan-1-ol (0.25 g) and (1S)-1-[1-(3,4-dichlorophenyl)cyclobutyl]-2-(piperidin-1-yl)ethan-1-ol (0.25 g) by preparative chiral HPLC, using the conditions below:

Column: Chiralpak-AD-H (250×30; 5.0 μm)

Mobile phase: Hexane: IPA: isopropylamine (95:05:0.1%)

Flow rate: 30 ml/min

UV: 225 nm

Diluent: Mobile phase

Loading: 25 mg/inj

Stacking: 15 min

Compounds 13 and 14 were prepared from the free bases using the synthetic protocol described for Compound 5.

Compound 13: LC-MS: m/z=328.1 [M+H]⁺. ¹H NMR (300 MHz, DMSOd₆) δ 9.32 (bs, 1H), 7.58 (d, J=8.3 Hz, 1H), 7.37 (d, J=2.1 Hz, 1H), 7.15 (dd, J=8.3, 2.1 Hz, 1H), 5.98 (d, J=5.1 Hz, 1H), 4.29 (dd, J=10.0, 4.9 Hz, 1H), 3.42 (dd, J=25.2, 10.3 Hz, 2H), 2.97 (dd, J=13.1, 6.1 Hz, 1H), 2.80 (d, J=10.8 Hz, 2H), 2.31 (p, J=9.6, 8.2 Hz, 4H), 2.14 (q, J=9.7 Hz, 1H), 1.99 (q, J=8.9, 8.5 Hz, 1H), 1.86-1.54 (m, 6H), 1.44-1.15 (m, 1H).

Compound 14: LC-MS: m/z=328.2 [M+H]⁺. ¹H NMR (300 MHz, DMSOd₆) δ 9.19 (bs, 1H), 7.58 (d, J=8.3 Hz, 1H), 7.37 (d, J=2.1 Hz, 1H), 7.15 (dd, J=8.4, 2.1 Hz, 1H), 5.99 (d, J=5.1 Hz, 1H), 4.27 (dd, J=9.7, 5.1 Hz, 1H), 3.33 (d, 2H), 2.95 (dd, J=6.6 Hz, 1H), 2.81 (m, J=10.3 Hz, 2H), 2.42-2.22 (m, 4H), 2.15 (d, J=9.6 Hz, 1H), 2.00 (q, 1H), 1.72 (q, J=23.1, 17.2 Hz, 6H), 1.30 (m, 1H).

### Example 14

### Synthesis of 1-[2-oxo-2-(1-phenylcyclobutyl)ethyl]piperidin-1-ium chloride (Compound 68)

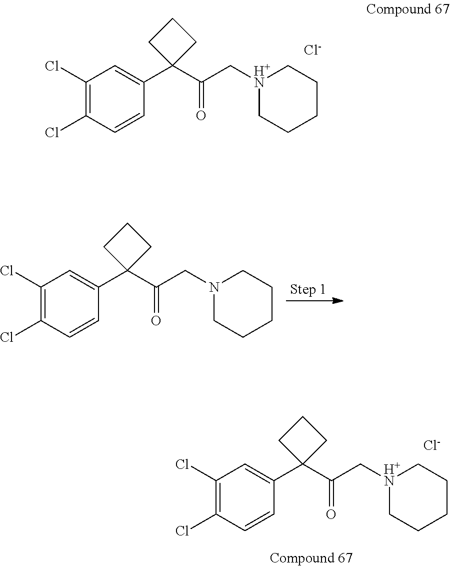

### Step 1: Synthesis of 1-[2-oxo-2-(1-phenylcyclobutyl)ethyl]piperidin-1-ium chloride

To a stirred solution of 1-(1-(3,4-dichlorophenyl)cyclopentyl)-2-(piperidin-1-yl)ethan-1-one (200 mg, 0.58 mmol) in 1,4-dioxane (2 mL) was added 4M HCl in dioxane (2 mL) at 0° C. The mixture was stirred at room temperature for 16 h then concentrated to dryness and washed with Et₂O (10 mL), EtOAc (10 mL). The organic solvent was decanted and the resulting solid was dried under vacuum to afford the title compound (160 mg, 72.7% yield) as an off white solid. LC-MS: m/z=328.3 [M+H]⁺. ¹H NMR (400 MHz, DMSOd₆) δ 9.57 (bs, 1H), 7.69 (d, J=8.4 Hz, 1H), 7.56 (d, J=2.2 Hz, 1H), 7.26 (dd, J=8.5, 2.2 Hz, 1H), 4.24 (d, J=5.1 Hz, 2H), 3.30 (d, J=32.5 Hz, 2H), 2.91-2.69 (m, 4H), 2.50 (p, J=1.9 Hz, 2H), 1.97-1.78 (m, 2H), 1.70 (dt, J=24.8, 9.7 Hz, 5H), 1.29 (t, J=11.8 Hz, 1H).

### Example 15

### Synthesis of Compounds 66, 68 and 69

The following compounds were synthesized using the same general synthetic protocols to those described in Example 67.

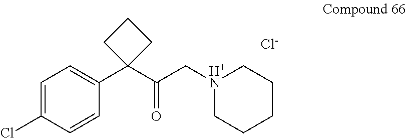

LC-MS: m/z=292.2 [M+H]⁺. ¹H NMR (400 MHz, DMSOd₆) δ 9.74 (bs, 1H), 7.72-7.38 (d, 2H), 7.40-7.16 (d, 2H), 4.19 (d, J=4.9 Hz, 2H), 3.47 (s, 2H), 3.26 (d, J=11.8 Hz, 2H), 3.06-2.64 (m, 4H), 2.42-2.29 (m, 2H), 2.03-1.74 (m, 2H), 1.74 (m, 3H), 1.27 (dt, J=15.1, 8.0 Hz, 1H).

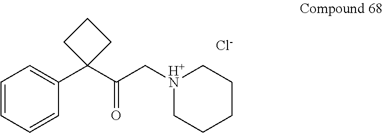

LC-MS: m/z=257.9 [M+H]⁺. ¹H NMR (400 MHz, DMSOd₆) δ 9.68 (bs, 1H), 7.44 (t, J=7.6 Hz, 2H), 7.41-7.12 (m, 3H), 4.17 (d, J=4.7 Hz, 2H), 3.24 (d, J=11.6 Hz, 2H), 2.98-2.68 (m, 4H), 2.51-2.35 (m, 2H), 2.03-1.78 (m, 2H), 1.79-1.53 (m, 5H), 1.28 (dt, J=12.8, 7.5 Hz, 1H).

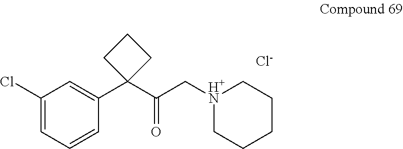

LC-MS: m/z=292.1 [M+H]⁺. ¹H NMR (400 MHz, DMSOd₆) δ 9.55 (bs, 1H), 7.46 (d, J=7.7 Hz, 1H), 7.42 (dt, J=8.2, 1.5 Hz, 1H), 7.37 (t, J=2.0 Hz, 1H), 7.24 (td, 1H), 4.23 (d, J=5.0 Hz, 2H), 3.31 (d, J=40.3 Hz, 2H), 2.89-2.66 (m, 4H), 2.51 (p, J=1.9 Hz, 2H), 1.98-1.80 (m, 2H), 1.79-1.62 (m, 5H), 1.30 (m, 1H).

### Example 16

### In Vitro Assays

### Measurement of DAT, NET and SERT Receptor Binding Activity

Affinity of the compounds for monoamine transporters was determined by in vitro radioligand binding assays using cell membrane preparations derived from HEK293 cell lines stably expressing human recombinant DAT, NET or SERT receptor (Suven Life Sciences, Hyderabad, India).

Stable DAT cell lines were generated following procedures described in Eshelman et al., Molecular Pharmacology 45, 312-316; 1994. Stable NET cell lines were generated following procedures described in Pacholczyk et al., Nature 350, 350-354, 1991 and Galli et al., Journal Exp. Biol. 198, 2197-2212, 1995. Stable SERT cell lines were generated following procedures described in Ramamoorthy et al., Proc Natl Acad Sci USA 90, 2542-246, 1993.

Cell membranes were obtained from the above-referenced cell lines by manually homogenizing previously frozen cell pellets in Tris-HCl buffer and serially centrifugating and re-extracting the preparations according procedures described in Subbu et al., Journal Pharmacol ExpTher 327, 982-990, 2008.

A scintillation proximity assay was used to measure receptor binding. Homogenized membrane preparations (final protein concentration 8-15 ug/well) were pre-incubated with WGA PVT SPA beads (0.5 mg/well) for 5 minutes. Binding was initiated by adding high affinity ligands and test compounds (0.1 nM to 10 μM) or reference/positive control ligands to the membrane-bead complex. Plates were incubated for three hours and raw data counts recorded using a liquid scintillation counter (MicroBetaTriLux Counter, Perkin Elmer). Inhibition constants (Ki) were calculated using GraphPad Prism software (version 4.0).

SERT receptor binding was determined using procedures described in Owens et al., The Journal of Pharmacology and Experimental Therapeutics 283:1305-1322, 1997. A mixture of [3H]citalopram (N-methyl-[3H]citalopram, Perkin Elmer) and bead-membrane complex was added to wells containing test compound. Non-specific binding was determined using wells containing venlafaxine hydrochloride (100 uM, Sigma). Total radioligand was determined using assay buffer containing 1% DMSO in the presence of [3H]citalopram. SERT Ki values were calculated using GraphPad Prism software (version 4.0).

NET receptor binding was determined using procedures described in Mason et al., The Journal of Pharmacology and Experimental Therapeutics 323:720-729, 2007; and Eshleman et al., The Journal of Pharmacology and Experimental Therapeutics289:877-885, 1999. A mixture of [3H]nisoxetine hydrochloride (N-methyl-[3H]nisoxetine, Perkin Elmer) and bead-membrane complex was added to wells containing test compound. Wells containing unlabeled nisoxetine (100 uM, Sigma) were used to define non-specific NET binding. Total radioligand was defined using assay buffer containing 1% DMSO in the presence of [3H]nisoxetine. NET Ki values were calculated using GraphPad Prism software (version 4.0).

DAT receptor binding was determined using procedures described in Skolnick et al., European Journal of Pharmacology, 461(2-3):99-104, 2003; and Eshleman et al., The Journal of Pharmacology and Experimental Therapeutics 289:877-885, 1999. A mixture of [3H]WIN-35428 (N-methyl 3H-WIN-35428, Perkin Elmer) and bead-membrane complex was added to wells containing test compound. Wells containing nomifensine maleate (10 mM, Sigma) were used to define non-specific SERT binding. Total radioligand was defined by assay buffer containing 1% DMSO in the presence of [3H]WIN-35428. DAT Ki values were calculated using GraphPad Prism software (version 4.0).

Table 2 below includes the DAT, NET and SERT Ki (nM) results of tested compounds. As listed in Table 2 below, numeral “1” indicates a Ki value of <10 nM; “2” indicates a Ki value of 11-100 nM; “3” indicates a Ki value of 101-500 nM; and “4” indicates a Ki value of >500 nM.

TABLE 2

| Compound No. | DAT Ki | NET Ki | SERT Ki |
|---|---|---|---|
| 1 | 3 | 4 | 4 |
| 5 | 4 | 4 | 4 |
| 6 | 4 | 4 | 4 |
| 7 | 2 | 3 | 4 |
| 8 | 4 | 4 | 4 |
| 9 | 4 | 4 | 4 |
| 10 | 3 | 4 | 4 |
| 66 | 4 | 4 | 4 |
| 67 | 2 | 4 | 3 |
| 68 | 4 | 4 | 4 |
| 2 | 4 | 4 | 4 |
| 11 | 4 | 4 | 4 |
| 3 | 2 | 2 | 3 |
| 4 | 4 | 4 | 4 |
| 12 | 4 | 4 | 4 |
| 69 | 4 | 4 | 4 |
| 13 | 2 | 2 | 4 |
| 14 | 2 | 3 | 4 |
| 71 | 2 | 3 | 4 |
| 15 | 2 | 2 | 3 |
| 60 | 2 | 3 | 3 |
| 16 | 2 | 3 | 4 |
| 61 | 2 | 4 | 4 |
| 17 | 2 | 3 | 4 |
| 18 | 2 | 2 | 4 |
| 19 | 2 | 2 | 3 |
| 20 | 2 | 2 | 3 |
| 59 | 4 | 4 | 4 |
| 21 | 1 | 2 | 4 |
| 22 | 1 | 3 | 4 |
| 23 | 1 | 3 | 4 |
| 24 | 1 | 4 | 3 |
| 25 | 2 | 2 | 3 |
| 27 | 2 | 3 | 4 |
| 29 | 2 | 3 | 4 |
| 30 | 2 | 4 | 4 |
| 31 | 1 | 2 | 2 |
| 32 | 1 | 3 | 2 |
| 33 | 1 | 3 | 4 |
| 34 | 2 | 2 | 3 |
| 35 | 2 | 3 | 4 |
| 36 | 2 | 3 | 3 |
| 38 | 1 | 2 | 3 |
| 39 | 3 | 3 | 4 |
| 42 | 2 | 3 | 3 |
| 47 | 2 | 2 | 3 |
| 48 | 2 | 2 | 3 |

### Example 17

### In Vivo Assays

Animals Used in the Study:

Male Sprague-Dawley rats (10-12 weeks, 270±30 g, RCC Laboratories India Private Limited, Hyderabad, India) were housed individually in round bottom Plexiglas bowl in conditions of constant temperature (21±3° C.) and humidity (30-70%), under a 12 h light/dark cycle with food and water ad libitum. The animals were acclimatized for a period of five days under test conditions.

Sterotaxic Surgical Procedures:

Pre-anesthetic medications (bupivacaine, adrenaline, 0.1 mL, s.c.) were administered at the site of incision, 5 min prior to the surgery and post-anesthetic medications (meloxicam 1.0 mg/kg, i.m. and gentamicin 16.0 mg/kg, i.m.) were administered after surgery.

Rats were anesthetized with gaseous administration of isoflurane and mounted in a stereotaxic apparatus (Stoelting) with the incisor bar set at 3.2 mm below the horizontal plane passing through the interaural line. Co-ordinates were taken according to Paxinos and Watson (1998) with reference points taken from the bregma and vertical from the skull. Holes were drilled for anchor screws and for placement of a guide cannula into the prefrontal cortex (AP +3.2 mm, ML −0.5 mm, DV −1.0 mm) or nucleus accumbens (AP +2.0 mm, ML −1.5 mm, DV −6.0 mm) or striatum (AP +3.2 mm, ML −0.5 mm, DV −1.0). Cannulae were secured to the skull using dental cement (DENTALON® plus) and anchor screws (BAS, IN, USA). The wound was sutured and the animals were allowed to recover for a minimum of 5 days in a round bottom Plexiglas bowl (BAS, IN, USA) with free access to water and feed.

All stereotaxic surgery was conducted in a room sterilized with UV lamps. Only animals without any visible signs of illness were used for the studies and the Microdialysis studies were conducted on fully recovered rats.

Formulation of test compound: Test compound formulations were prepared freshly on the day of study.

Microdialysis:

Approximately 15 h prior to the microdialysis experiment, rats were connected to a dual quartz lined two-channel liquid swivel (Instech, UK) on a counter balance lever arm, which allowed unrestricted movement of the freely moving animal. Pre-equilibrated microdialysis probes with 4 mm dialysis membrane (BR-4, 4 mm, BAS) for prefrontal cortex or striatum and 2 mm dialysis membrane (CMA/11, 2 mm, CMA Microdialysis) for nucleus accumbens were inserted snugly into the guide cannula. The input tube of the dialysis probe was connected to a syringe pump (BeeHive and BabyBee, BAS) and the output tube connected to a refrigerated fraction collector (HoneyComb, BAS). Animals were fasted overnight (approximately 14 h prior to dosing), and food was returned to the cages at 2 h post-dose.

On the day of study, the probe was perfused at a constant flow rate of 1.0 μL/min with artificial cerebrospinal fluid (aCSF; NaCl 150 mmol, KCl 3.0 mmol, MgCl₂ 0.9 mmol, CaCl₂ 2H₂O 1.7 mmol pH 6.2). After the initiation of perfusion, a stabilization period of 2 hours was maintained and four basal samples were collected at 30 min intervals. Test compound or vehicle was administered via oral gavage or intraperitoneal injection, and dialysate samples were collected at 30 min intervals for up to 24 hours using a refrigerated fraction collector (HoneyComb, BAS). Following collection, dopamine, norepinephrine and serotonin levels were quantified in the dialysate samples.

Histology:

After completion of the dialysis experiment, animals were sacrificed with carbon-dioxide asphyxiation for histological observations. Rat brains were fixed in 10% formalin and later sliced in 50 μM sections on a cryostat (Leica), stained with cresyl violet (Sigma-Aldrich). Photomicrographs taken from representative sections from each animal to confirm probe placement. Data from animals with incorrect probe placement was excluded from the analysis.

Analytical Procedures:

The catecholamine neurotransmitters dopamine (DA), norepinephrine (NE), and serotonin (5-HT) were analysed in dialysates according to Nirogi et al., Journal of Chromatography B, 913-914, p. 41-47, 2013. Briefly, samples were subjected to derivatization with dansyl chloride and monitored at m/z 853.1-170.1, m/z 869.2-170.1 and m/z 643.3-170.1 to determine dansylated DA, NE and 5-HT, levels respectively. The analytes were quantified using a triple quadrupole tandem mass spectrometer in positive ionization mode and an atmospheric pressure ionization source. A gradient elution method was used to separate the analytes from interference on an Agilent Poroshell 120 EC-C18 outer porous micro particulate column. The test samples were quantified relative to a calibration curve for each transmitter, prepared using artificial cerebrospinal fluid, over a concentration range from 0.066 to 14.835 nM.

Data Analysis: Absolute values (in nmol/L) of dopamine, norepinephrine and serotonin were converted into % change±SEM from mean basal value with 100% defined as the average of four pre-dose values. Individual basal values, more than ±50% of the mean basal value were excluded from computation of mean basal values. In addition, incorrect probe placement and basal values below the lower the limit of quantification for any analyte were used as criteria to reject the data from individual animals. In addition, individual data-points which were 2-fold different from both the previous and the next following sample were considered outliers and excluded from further calculations. Excluded data points are shown in bold on the data tables.

Typically, compounds that increase dopamine or norepinephrine level or both levels by 75% or more (e.g., 100-900%) in the striatum, nuculeus accumbens and especially the prefrontal cortex, relative to baseline neurotransmitter levels in untreated subject such as an animal, are suitable candidates for treating or preventing CNS diseases or conditions.

### INCORPORATION BY REFERENCE

The entire disclosure of each of the patent documents and scientific articles referred to herein is incorporated by reference for all purposes.

### EQUIVALENTS

The disclosure can be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

What is claimed is:

1. A compound of Formula (I): 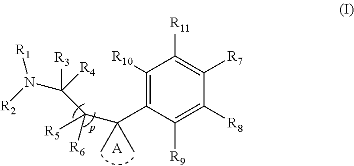
or a pharmaceutically acceptable salt thereof, wherein
ring A is C₃-C₆ cycloalkyl optionally substituted with one or more C₁-C₃ alkyl;
each of R₁ and R₂ independently, is H or R_{S1}, in which R_{S1} is C₁-C₆ alkyl, C₂-C₆ alkenyl, C₂-C₆ alkynyl, or C₃-C₈ cycloalkyl, and R_{S1} is optionally substituted with one or more substituents selected from the group consisting of halo, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl; and at least one of R₁ and R₂ is not H; or
R₁ and R₂, together with the nitrogen atom to which they are attached, form a 4 to 12-membered saturated heterocycloalkyl ring having 0 to 2 additional heteroatoms, and the 4 to 12-membered saturated heterocycloalkyl ring is optionally substituted with one or more substituents selected from the group consisting of halo, hydroxyl, cyano, C₁-C₆ alkyl, C₁-C₆ haloalkyl, C₂-C₆ alkenyl, C₂-C₆ alkynyl, C₁-C₆ alkoxyl, C₁-C₆ haloalkoxyl, amino, mono-C₁-C₆ alkylamino, di-C₁-C₆ alkylamino, C₃-C₈ cycloalkyl, or 4 to 12-membered heterocycloalkyl;
each of R₃ and R₄ independently, is H, C₁-C₆ alkyl, C₂-C₆ alkenyl, or C₂-C₆ alkynyl; or
R₃ and R₄, together with the carbon atom to which they are attached, form C₃-C₆ cycloalkyl;
R₅ is OR_{S2}, in which R_{S2} is H or C₁-C₆ alkyl;
R₆ is H or C₁-C₆ alkyl; or
R₅ and R₆, together with the carbon atom to which they are attached, form C═O;
each of R₇, R₈, R₉, R₁₀, and R₁₁, independently, is -Q-T, in which Q is a bond or C₁-C₃ alkyl linker optionally substituted with halo, cyano, hydroxyl or C₁-C₆ alkoxy, and T is H, halo, hydroxyl, C(O)OH, cyano, azido, or R_{S3}, in which R_{S3} is C₁-C₃ alkyl, C₂-C₆ alkenyl, C₂-C₆ alkynyl, C₁-C₆ alkoxyl, C₁-C₆ thioalkyl, C(O)OC₁-C₆ alkyl, C(O)NH₂, SO₂C₁-C₆ alkyl, SO₂C₆-C₁₀ aryl, SO₂NH₂, C(O)NH(C₁-C₆ alkyl), C(O)N(C₁-C₆ alkyl)₂, SO₂NH(C₁-C₆ alkyl), SO₂N(C₁-C₆ alkyl)₂, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl; or
R₇ and R₈, together with the carbon atoms to which they are attached, form phenyl or a 5- or 6-membered heteroaryl having 1 to 3 heteroatoms; or R₇ and R₁₁, together with the carbon atoms to which they are attached, form phenyl or a 5- or 6-membered heteroaryl having 1 to 3 heteroatoms; or R₈ and R₉, together with the carbon atoms to which they are attached, form phenyl or a 5- or 6-membered heteroaryl having 1 to 3 heteroatoms; or R₁₀ and R₁₁, together with the carbon atoms to which they are attached, form phenyl or a 5- or 6-membered heteroaryl having 1 to 3 heteroatoms; and
p is 0 or 1, provided that
(i) when p is 0, then ring A is optionally substituted C₃-C₅ cycloalkyl or substituted cyclohexyl, wherein when ring A is unsubstituted cyclobutyl, then both R₃ and R₄ are H;
(ii) when p is 0 and NR₁R₂ is unsubstituted piperidin-1-yl, then ring A is unsubstituted cyclobutyl or substituted C₃-C₆ cycloalkyl, or at least one of R₇, R₈, R₉, R₁₀, and R₁₁ is not H; and
(iii) when p is 1 and ring A is unsubstituted cyclobutyl, then R₅ is OR_{S2}, or R₅ and R₆, together with the carbon atom to which they are attached, form C═O.

2. The compound of claim 1, wherein R₃ and R₄ are both H, and R₆, R₉, R₁₀ and R₁₁ are all H.

3. (canceled)

4. The compound of claim 2, wherein the compound is of Formula (Ia):

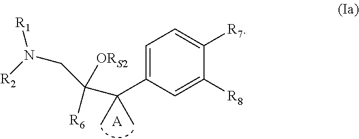

5. The compound of claim 4, wherein R_{S2} is H.

6. The compound of claim 2, wherein the compound is of Formula (Ib): 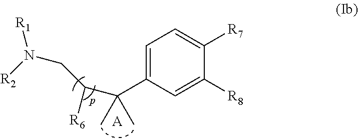
and p is 0.

7. (canceled)

8. The compound of claim 2, wherein the compound is of Formula (Ic):

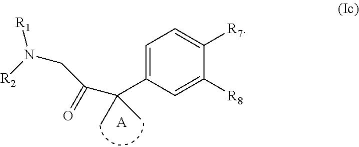

9. The compound of claim 1, wherein ring A is unsubstituted.

10. The compound of claim 1, wherein ring A is substituted with one or more C₁-C₃ alkyl.

11. The compound of claim 10, wherein ring A is substituted with one C₁-C₃ alkyl.

12. The compound of claim 1, wherein ring A is optionally substituted C₄-C₅ cycloalkyl.

13. The compound of claim 12, wherein the compound is of Formula (Ia) or (Ib) and ring A is unsubstituted cyclobutyl.

14. The compound of claim 12, wherein the compound is of Formula (Ic) and ring A is unsubstituted cyclopentyl.

15. The compound of claim 12, wherein the compound is of Formula (Ic) and ring A is optionally substituted cyclobutyl.

16. The compound of claim 2, wherein each of R₇ and R₈, independently, is H, halo, or cyano.

17. The compound of claim 16, wherein at least one of R₇ and R₈ is halo.

18. The compound of claim 17, wherein each of R₇ and R₈ is chloro.

19. The compound of claim 16, wherein R₇ and R₈, together with the carbon atoms to which they are attached, form phenyl, pyridyl, pyrrolyl, furanyl, thienyl, thiazolyl, oxazolyl, imidazolyl, pyrazolyl, isoxazolyl, triazolyl, oxadiazolyl, pyridazinyl, pyrazinyl, or pyrimidyl.

20. The compound of claim 2, wherein one of R₁ and R₂ is H and the other is C₁-C₆ alkyl optionally substituted with halo or is C₃-C₈ cycloalkyl optionally substituted with C₁-C₆ alkyl.

21. The compound of claim 20, wherein the other of R₁ and R₂ is isopropyl or t-butyl, optionally substituted with one or more halo groups.

22. The compound of claim 1, wherein one of R₁ and R₂ is C₁-C₆ alkyl optionally substituted with halo and the other is C₁-C₆ alkyl optionally substituted with halo or is C₂-C₆ alkynyl or C₃-C₈ cycloalkyl optionally substituted with C₁-C₆ alkyl.

23. The compound of claim 1, wherein R₁ and R₂, together with the nitrogen atom to which they are attached, form an optionally substituted 5 to 8-membered saturated heterocycloalkyl ring having 0 to 2 additional heteroatoms.

24. The compound of claim 23, wherein the 5 to 8-membered saturated heterocycloalkyl ring is unsubstituted.

25. The compound of claim 23, wherein the 5 to 8-membered saturated heterocycloalkyl ring is substituted with 1-3 substituents selected from the group consisting of halo, cyano, C₁-C₆ alkyl, C₁-C₆ haloalkyl, and C₁-C₆ alkoxyl.

26. The compound of claim 23, wherein the 5 to 8-membered saturated heterocycloalkyl ring is substituted with one or two substituents selected from the group consisting of fluoro, cyano, CH₃, CH₂CH₃, CF₃, and OCH₃.

27. The compound of claim 23, wherein the 5 to 8-membered saturated heterocycloalkyl ring is selected from piperidin-1-yl, pyrrolidin-1-yl, azepane-1-yl, morpholin-4-yl, 3-azabicyclo[3.2.1]octan-3-yl, 2-azabicyclo[2.2.1]heptan-2-yl, and 2-oxa-5-azabicyclo[2.2.1]heptan-5-yl.

28. The compound of claim 27, wherein the 5 to 8-membered saturated heterocycloalkyl ring is piperidin-1-yl; and when substituted with one or more fluoro is substituted at the 3-, 4-, or both positions with fluoro; when substituted with one or more C₁-C₆ alkoxyl, is substituted at the 3-, 4-, or both positions with C₁-C₆ alkoxyl; and when substituted with one or more cyano, is substituted at the 3-, 4-, or both positions with cyano.

29. The compound of claim 27, wherein the 5 to 8-membered saturated heterocycloalkyl ring is selected from morpholin-4-yl, azepane-1-yl, 3-azabicyclo[3.2.1]octan-3-yl, 2-azabicyclo[2.2.1]heptan-2-yl, and 2-oxa-5-azabicyclo[2.2.1]heptan-5-yl.

30. The compound of claim 29, wherein the 5 to 8-membered saturated heterocycloalkyl ring is unsubstituted 2-oxa-5-azabicyclo[2.2.1]heptan-5-yl.

31.-32. (canceled)

33. A compound selected from Compound 3, 19, 20, 21, 27, 29, 32 and 61.

34.-48. (canceled)
